(19) United States
(12) Patent Application Publication
(10) Pub. No.: US 2025/0127933 A1
(43) Pub. Date: Apr. 24, 2025

(54) Method of verifying effectiveness and stability of linker in Drug Delivery system and Structure therefor

(71) Applicant: INDUSTRY-ACADEMIC COOPERATION FOUNDATION OF SUNCHON NATIONAL UNIVERSITY, Suncheon-si (KR)

(72) Inventors: Dong Jo CHANG, Suncheon-si (KR); Sung-Tae YEE, Suncheon-si (KR)

(21) Appl. No.: 18/382,627

(22) Filed: Oct. 23, 2023

(51) Int. Cl. A61K 49/00; A61M 5/00

(52) U.S. Cl. CPC A61K 49/0041; A61K 49/0043; A61K 49/0052; A61M 5/00

(57) ABSTRACT

The present invention relates to a method and a structure for verifying the effectiveness of a linker in a ligand-targeted drug delivery system. The structure includes a fluorescent probe, which replaces a drug in the ligand-targeted drug delivery system and is linked to the linker whose effectiveness is to be verified. The fluorescent probe consists of a fluorophore which fluoresces when the linker in the ligand-targeted drug delivery system is cleaved, or consists of a combination of the fluorophore and a spacer. According to the structure and method of the present invention, it is possible to easily and simply confirm whether the linker in the ligand-targeted drug delivery system effectively releases the drug by being stably cleaved in vivo, thereby reducing the development period and cost of ligand-targeted drugs.

### TECHNICAL FIELD OF THE INVENTION

The present invention relates to a method for verifying the effectiveness of a linker in a drug delivery system and a structure for the method. Specifically, the present invention relates to a method and a structure for verifying whether a linker in drug delivery systems (DDSs), such as prodrugs, antibody-drug conjugates, or ligand-targeted drugs, effectively releases an active drug by being cleaved in a disease-selective or protein-selective manner in vivo and whether the linker is stable in plasma.

### BACKGROUND ART OF THE INVENTION

To overcome the limitations of conventional chemotherapy, such as toxicity, ligand-targeted drugs (LTDs) have been developed to enhance the selective localization of small-molecule drugs to tumors. In an LTD, a targeting ligand as a vehicle for specific delivery to cancer cells is conjugated with a therapeutic or imaging agent via a spacer and cleavable or non-cleavable linker. Monoclonal antibodies, peptides, and small molecules specific to malignant antigens are considered vehicles to selectively deliver cargo, such as cytotoxic or imaging agents, to target cells.

Antibody-drug conjugates (ADCs) are one of the best-characterized strategies for LTDs. Since the discovery and successful landing of brentuximab vedotin (Adcetris®, Seattle Genetics, 2011) against relapsed Hodgkin lymphoma and systemic anaplastic large cell lymphoma in the market [1-3]. 10 ADCs have been approved by the United States Food and Drug Administration, and there are numerous ADCs being explored in ongoing clinical trials [4-12].

Typical ADCs comprise a humanized or fully human monoclonal antibody (mAb) targeting an antigen specifically overexpressed in tumor cells, cytotoxic payloads, and a suitable chemical linker for the covalent conjugation of drugs to antibodies. ADCs recognize and bind to highly expressed antigens on the cell surface of target cancer cells, and the ADC-antigen complex is internalized via endocytosis, followed by the release of cytotoxic drugs by lysosomal degradation.

The linker is a crucial component that determines various attributes of an ADC required for clinical use, including the efficacy and therapeutic index in terms of pharmacokinetics, pharmacodynamics, and toxicity. As encountered in the history of the development of many ADCs, instability of the linker leads to the premature release of a cytotoxic payload in the plasma during blood circulation or in other tissues, which may result in higher toxicity and lower efficacy [13-16].

For the successful development of ADC drugs, it is important to develop linkers that have sufficient plasma stability during blood circulation and selectively release the payloads via internalization into cancer cells. As demonstrated in the discovery of brentuximab vedotin, a valine-citrulline (Val-Cit) dipeptide linker was designed to provide sufficient plasma stability, which is conjugated to cytotoxic momomethyl auristatin E (MMAE) via a 4-aminobenzyl carbamate linkage (FIG. 1) [1-2]. Seattle Genetics first conjugated MMAE to cBr96 mAb using a dipeptide linker (Phe-Lys) containing a lysine residue for the recognition and cleavage by cathepsin B [2]. The Phe-Lys linker-conjugated ADC showed a half-life of 12.5 d in mice, whereas the Val-Cit linker-conjugated ADC has a half-life of 30 day [2,17]. As shown in the ADCs with enzymatic cleavable linkers, including brentuximab vedotin and recently approved trastuzumab deruxtecan (Enhertu®, AstraZeneca & Daiichi Sankyo, 2019), cytotoxic payloads in ADCs are generally conjugated to cleavable linkers through a self-eliminating p-aminobenzyl carbamate or carbonate linkage. The p-aminobenzyl group is a self-eliminating spacer that is generally used to spatially separate cytotoxic payloads and cleavable linkers for better recognition and cleavage by lysosomal peptidases [18-22].

The present inventors previously reported various xanthene-based fluorophores and activatable fluorescent probes that can release fluorophores and activate fluorescence under the activity of a specific enzyme. In previous studies, the present inventors synthesized “turn on” nitroreductase (NTR)-responsive fluorescent probes diversely linked to a 4-nitrobenzyl moiety via ether, carbonate, amine, and carbamate linkages [23,24].

Fluorescence emission by decomposition of the linker was used as a means for evaluating the stability at various pH values and temperatures, as well as the release of fluorophores under the NTR reaction. Based on such previous studies, the present inventors have designed and evaluated peptide-based cleavable linkers having sufficient plasma stability using activatable fluorescent probes and have found that these cleavable linkers can selectively release cytotoxic payloads in response to a tumor-specific enzyme, thereby completing the present invention.

### PRIOR ART DOCUMENTS

### Non-Patent Document

(1) Francisco, J. A.; Cerveny, C. G.; Meyer, D. L.; Mixan, B. J.; Klussman, K.; Chace, D. F.; Rejniak, S. X.; Gordon, K. A.; DeBlanc, R.; Toki, B. E. Blood 2003, 102, 1458-1465.
(2) Doronina, S. O.; Toki, B. E.; Torgov, M. Y.; Mendelsohn, B. A.; Cerveny, C. G.; Chace, D. F.; DeBlanc, R. L.; Gearing, R. P.; Bovee, T. D.; Siegall, C. B. Nat. Biotechnol. 2003, 21, 778-784.
(3) Senter, P. D.; Sievers, E. L. Nat. Biotechnol. 2012, 30, 631-637.
(4) Nejadmoghaddam, M. R.; Minai Tehrani, A.; Ghahremanzadeh, R.; Mahmoudi, M.; Dinarvand, R.; Zarnani, A. H. Avicenna. J. Med. Biotechnol. 2019, 11, 3-23.
(5) Bross, P. F.; Beitz, J.; Chen, G.; Chen, X. H.; Duffy, E.; Kieffer, L.; Roy, S.; Sridhara, R.; Rahman, A.; Williams, G. Clin. Cancer Res. 2001, 7, 1490-1496.
(6) Younes, A.; Bartlett, N. L.; Leonard, J. P.; Kennedy, D. A.; Lynch, C. M.; Sievers, E. L.; Forero Torres, A. N. Engl. J. Med. 2010, 363, 1812-1821.
(7) Ballantyne, A.; Dhillon, S. Drugs 2013, 73, 755-765.
(8) Richardson, N. C.; Kasamon, Y. L.; Chen, H.; de Claro, R. A.; Ye, J.; Blumenthal, G. M.; Farrell, A. T.; Pazdur, R. Oncologist 2019, 24, e180-187.
(9) Deeks, E. D. Drugs 2019, 79, 1467-1475.
(10) Hanna, K. S. Drugs 2020, 80, 1-7.
(11) Keam, S. J. Drugs 2020, 80, 501-508.
(12) Syed, Y. Y. Drugs 2020, 80, 1019-1025.
(13) Beck, A.; Goetsch, L.; Dumontet, C.; Corvaia, N. Nat. Rev. Drug Discov. 2017, 16, 315-337.
(14) Nolting, B. In Methods in Molecular Biology (Methods and Protocols), L., D., Ed.; Humana Press: Totowa, NJ., 2013, pp 71-100.
(15) Hughes, B. Nat. Rev. Drug Discov. 2010, 9, 665-667.
(16) Sau, S.; Alsaab, H. O.; Kashaw, S. K.; Tatiparti, K.; Iyer, A. K. Drug Discov. Today 2017, 22, 1547-1556.
(17) Ducry, L.; Stump, B. Bioconjug. Chem. 2010, 21, 5-13.
(18) Dal Corso, A.; Pignataro, L.; Belvisi, L.; Gennari, C. Chem. Eur. J. 2019, 25, 14740-14757.
(19) Alouane, A.; Labruere, R.; Le Saux, T.; Schmidt, F.; Jullien, L. Angew. Chem. Int. Ed. Engl. 2015, 54, 7492-7509.
(20) Greenwald, R. B.; Pendri, A.; Conover, C. D.; Zhao, H.; Choe, Y. H.; Martinez, A.; Shum, K.; Guan, S. J. Med. Chem. 1999, 42, 3657-3667.
(21) Zhang, D.; Le, H.; Cruz Chuh, J. d.; Bobba, S.; Guo, J.; Staben, L.; Zhang, C.; Ma, Y.; Kozak, K. R.; Lewis Phillips, G. D. Bioconjug. Chem. 2018, 29, 267-274.
(22) Jain, N.; Smith, S. W.; Ghone, S.; Tomczuk, B. Pharm. Res. 2015, 32, 3526-3540.
(23) More, K. N.; Lim, T. H.; Kim, S. Y.; Kang, J.; Inn, K. S.; Chang, D. J. Dyes Pigm. 2018, 151, 245-253.
(24) More, K. N.; Lim, T. H.; Kang, J.; Yun, H.; Yee, S. T.; Chang, D. J. Molecules 2019, 24, 3206.

### DISCLOSURE

### Technical Problem

An object of the present invention is to provide a method for verifying the effectiveness and stability of a linker in a drug delivery system.

Another object of the present invention is to provide a structure for verifying the effectiveness and stability of the linker.

### Technical Solution

LTDs have been of interest as a way to solve the safety and efficacy issues of chemotherapeutics, which are still widely used in clinical practice. The efficacy of LTDs such as ADCs is primarily determined by the activity of payloads, while the safety may be controlled by the stability and selectivity of linkers for conjugation, as well as the selectivity of targeting ligands for delivery. The selective release of payloads to target cells is a critical factor for successful LTDs with sufficient efficacy and minimal toxicity. In addition, in prodrugs, the stability and selectivity of the carrier (or linker) that selectively releases the payload to target cells are also critical factors in the development of prodrugs. Thus, it is essential to develop cleavable linkers with sufficient plasma stability during blood circulation that selectively releases payloads in target cells.

Accordingly, the present invention has been completed based on the finding that, although activatable fluorescent probes have no ligand for delivery, the release of fluorophores conjugated to peptide linkers from the activatable fluorescent probes may be easily detected and evaluated based on emitted fluorescence. As a specific example, an enzyme-responsive activatable fluorescent probe provides a simple and intuitive method for the evaluation of the inherent stability and drug release efficiency of cleavable linkers and a structure for the method (FIG. 2).

In one aspect, the present invention provides a structure for verifying the effectiveness and stability of a linker in drug delivery systems, including antibody-drug conjugates.

Specifically, the present invention provides a structure for verifying whether a cleavable linker in drug delivery systems, including antibody-drug conjugates in which a ligand, the cleavable linker, and a drug are linked together, is selectively cleavable in a specific disease or by a specific protein, is stable in vivo, and efficiently releaser the drug in vivo.

In the present invention, the term “drug delivery system (DDS)” refers to a drug delivery system having either a structure in which a vehicle (or linker) capable of cleavage at or near an in vivo target and a drug that exhibits efficacy by decomposition of the vehicle are linked together, or a structure in which a ligand that binds to an in vivo target, a cleavable linker, and a drug are linked together.

The in vivo target or target may be a specific protein, examples of which include, but are not necessarily limited to, CD33, CD30, HER2+, CD22, CD79b, Nectin 4, HER2, TROP-2, B-cell maturation antigen (BCMA), CD19, tissue factor (TF), folate receptor, and the like.

In the present invention, a prodrug is one of the above-described drug delivery systems and has a structure in which a vehicle (or linker) capable of cleavage at or near an in vivo target and a drug that is released by decomposition of the vehicle are linked together.

In the present invention, a ligand-targeted drug (LTD) is one of the above-described drug delivery systems and has a structure in which a ligand that binds to an in vivo target, a cleavable linker, and a drug are linked together. The drug exhibits efficacy by decomposition of the linker.

In the present invention, an antibody-drug conjugate (ADC) is a representative example of the ligand-targeted drug and is a drug delivery system in which an antibody that binds to an in vivo target, a cleavable linker, and a drug are linked together. Unless otherwise specified, in the present specification, the term “ligand-targeted drug” is intended to include the term “antibody-drug polymer (ADS)”.

The structure of the present invention is characterized by including a fluorescent probe, which replaces the drug in the drug delivery system and is linked to the linker whose effectiveness is to be verified.

The linker whose effectiveness is to be verified is a linker which is developed for use in drug delivery systems, including prodrugs, antibody-drug conjugates, and ligand-targeted drugs. The linker is a compound linked to the drug in prodrugs among drug delivery systems or is a compound that links the drug to the ligand in antibody-drug conjugates or ligand-targeted drugs among drug delivery systems. According to the present invention, it is verified whether the linker is stable in vivo (preferably in plasma) and is cleavable in vivo by a protease or peptidase present at or near an in vivo target, and whether the drug loaded in the drug delivery system may be efficiently released in vivo by the cleavage of the linker.

As a specific example, the linker may be an enzyme substrate that is cleaved by an enzyme present at or around an in vivo target. The enzyme substrate may be, for example, leucine that is degraded by leucine peptidase, without being necessarily limited thereto.

The fluorescent probe of the present invention may be a fluorophore that fluoresces when the linker in the drug delivery system is cleaved. The fluorophore may be linked directly to the linker whose effectiveness is to be verified. Preferably, the fluorophore may be linked via a spacer to the linker whose effectiveness is to be verified.

Although the linkage between the fluorescent probe and the linker whose effectiveness is to be verified may vary depending on the types of fluorescent probe and linker whose effectiveness is to be verified, examples thereof include an ether linkage, an ester linkage, an amide linkage, a carbonate linkage, a carbamate linkage, and the like.

The fluorophore of the present invention may be any fluorescent substance that can fluoresce when the linker is cleaved in vivo, and examples thereof include, but are not necessarily limited to, coumarin derivatives such as 7-amino-methylcoumarin (AMC); xanthine derivatives such as rhodamine, rhodol, fluorescein, and eosin; cyanine derivatives such as cyanine, indocarbocyanine, oxacarbocyanine, thiacarbocyanine, and merocyanine; and squaraine derivatives.

The spacer, a compound that links the fluorophore to the linker, is a compound that self-eliminates when the linker is cleaved. Examples of the spacer include, but are not necessarily limited to, a p-aminobenzyl spacer, etc. The p-amino benzyl spacer may be linked via an ether linkage, an ester linkage, an amide linkage, a carbonate linkage, a carbamate linkage, or the like to the linker whose effectiveness is to be verified.

As a specific example, the fluorescent probe of the present invention may be a xanthine derivative having a structure represented by Formula 43 or 44 below.

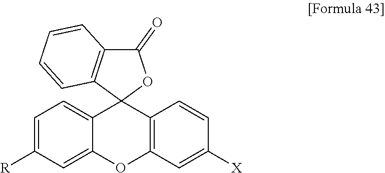

the R is MeO— or Et₂N—, and
the X is —NH₂,

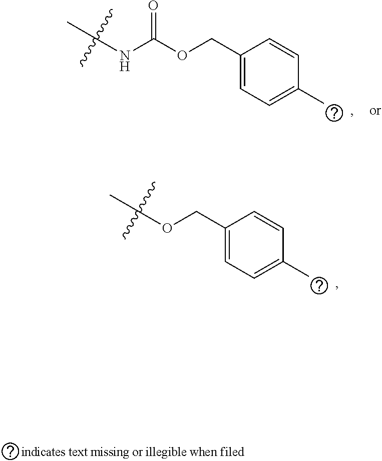

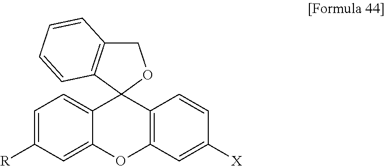

The R is MeO— or Et₂N—, and
the X is —NH₂,

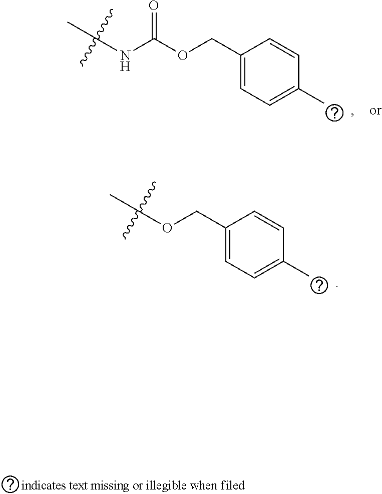

More specifically, the fluorescent probe of the present invention, which consists only of a fluorophore that is linked directly to the linker, may be any one of the compounds of Formulas 43 and 44 wherein R is MeO— or Et₂N— and X is —NH₂. As an example, a structure in which the fluorescent probe consisting only of a fluorophore is linked to leucine as the linker is a compound of Formula 1, a compound of Formula 2, a compound of Formula 7, or a compound of Formula 8 (see C-2 and Scheme S2 described in the Examples).

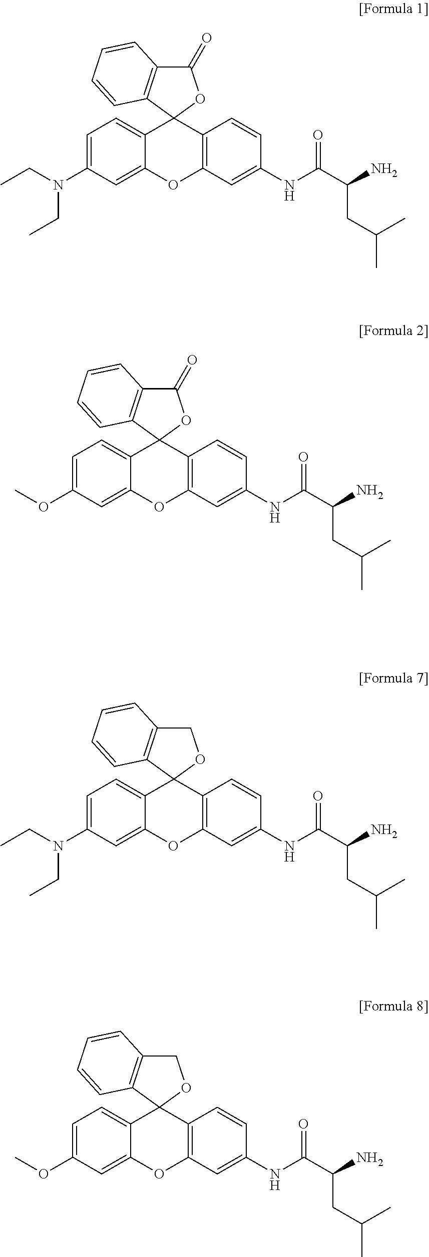

More specifically, the fluorescent probe of the present invention, which consists of the fluorophore and the spacer, may be any one of compounds of Formulas 43 and 44 wherein R is MeO— or Et₂N— and X is

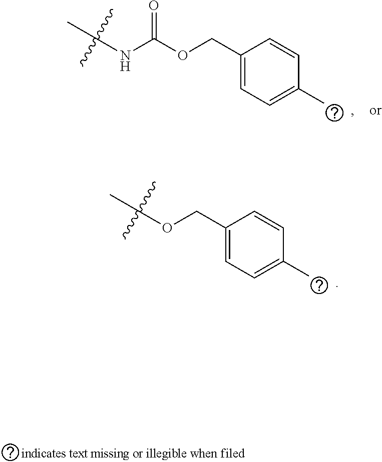

As an example, a structure in which the fluorescent probe consisting of the fluorophore and the spacer is linked to leucine as the linker is a compound of Formula 3, a compound of Formula 4, a compound of Formula 5, a compound of Formula 6, a compound of Formula 9, a compound of Formula 10, a compound of Formula 11, or a compound of Formula 12 (see C-3, Scheme S3 and C-4, and Scheme S4 described in the Examples).

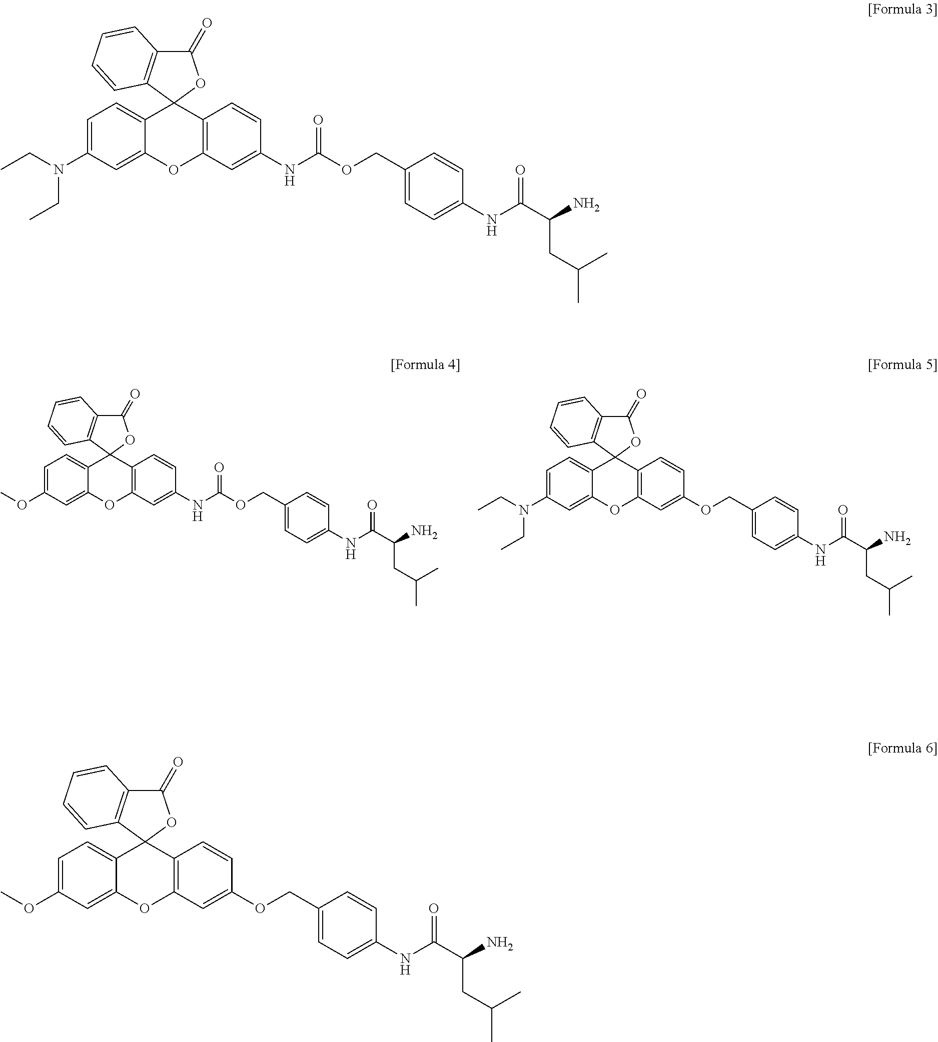

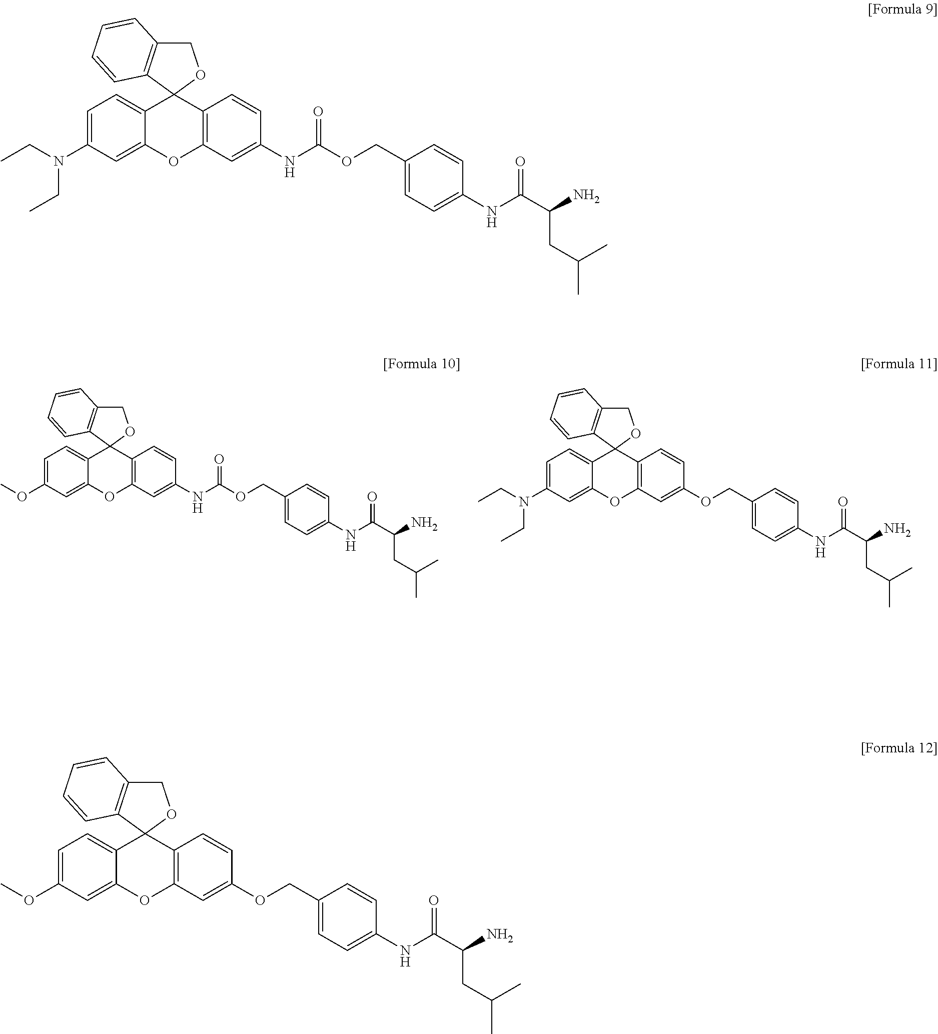

As examples of the structure of the present invention, various LAP-responsive fluorescent probes containing leucine, an LAP substrate, were prepared and tested. As a result, it was found that the fluorescent probes showed stable fluorescence due to cleavage of leucine at pH 2 to 12, which is the in vivo pH condition, and showed stable fluorescence even in the temperature range of 25 to 45° C. In addition, it was found that the fluorescent probes exhibited fast or slow kinetics depending on the type of fluorescent probe prepared, released fluorophores by lysosomal LAP rather than extracellular LAP, emitted fluorescence only by LAP without interference from other intracellular bioanalytes, and were stable even in an ex vivo plasma stability test.

Therefore, the structure of the present invention probes may be used as a platform for the development of cleavable linkers for drug delivery systems that effectively drugs and are sufficiently stable during blood circulation.

In another aspect, the present invention provides a method for verifying the effectiveness of a linker in a drug delivery system.

Specifically, the present invention provides a method for verifying the effectiveness, stability, in vivo stability, in vivo cleavability, and in vivo drug release efficiency of a cleavable linker in drug delivery systems, including prodrugs in which the cleavable linker and a drug are linked together, and in antibody-drug conjugates or ligand-targeted drugs in which a ligand, the cleavable linker and a drug are linked together.

The method for verifying the effectiveness and stability of a linker according to the present invention includes steps of: preparing a structure for verifying the effectiveness of a linker, developed for use in ligand-targeted drug delivery systems, including antibody-drug conjugates, by linking a fluorescent probe to the linker; and confirming whether the structure emits fluorescence by cleavage of the linker inside or outside a cell.

The step of preparing a structure for verifying the effectiveness of a linker is a step of linking a fluorescent probe to a linker developed for use in the above-described drug delivery systems, including prodrugs, antibody-drug conjugates, and ligand-targeted drugs, that is, a linker whose effectiveness is to be verified.

The linker whose effectiveness is to be verified, the fluorescent probe, and the linkage therebetween are as described above.

The step of confirming whether the structure emits fluorescence by cleavage of the linker inside or outside a cell is a step of confirming whether the fluorophore in the fluorescent probe emits fluorescence by in vivo or ex vivo cleavage of the linker whose effectiveness is to be verified, that is, the linker developed for use in a drug delivery system, and measuring the amount of the emitted fluorescence.

The cell may be a cell to which the ligand in a ligand-targeted drug system binds or a cell on which the drug acts. The “inside a cell” may refer to in vivo, and the “outside a cell” may refer to ex vivo.

Confirming whether the fluorophore emits fluorescence by cleavage of the linker inside the cell means confirming that the cleavage of the linker is caused by an intracellular substance (e.g., lysosomal LAP) rather than an extracellular substance (e.g., extracellular LAP). This confirmation may be an indicator for evaluating the efficiency of drug release by intracellular cleavage of the linker.

Confirming whether the fluorophore emits fluorescence by cleavage of the linker outside the cell may be an indicator for evaluating the stability of the linker depending on in vivo temperature and pH conditions.

In addition, confirming whether the fluorophore emits fluorescence by cleavage of the linker outside the cell may be an indicator to evaluate the stability and drug release efficiency of the linker by determining whether the cleavage of the linker is caused by the target substance in vivo or by another substance in vivo.

In addition, confirming the timing and amount of fluorescence emission by cleavage of the linker inside or outside the cell may be an indicator for evaluating the pharmacokinetics of the linker, the in vivo stability of the linker, and the half-life of the ligand-targeted drug including the linker.

Although confirmation of the fluorescence emission may vary depending on the type of fluorophore in the structure, it may be performed using a fluorescence detector such as a spectrophotometer.

According to the above-described method for verifying the effectiveness of a linker in a drug delivery system, it is possible to easily and simply evaluate the stability and drug release efficiency of a linker for use in a ligand-targeted drug delivery system, thereby dramatically reducing the development period and cost of drug delivery systems, including prodrugs, antibody-drug conjugates, and ligand-targeted drugs.

### Advantages Effects

According to the structure of the present invention and the method for verifying the effectiveness and stability of a linker using the same, it is possible to easily and simply confirm whether a linker in drug delivery systems, including prodrugs, antibody-drug conjugates, and ligand-directed drugs, is stable in vivo, is cleavable in vivo, and efficiently releases the drug in vivo, thereby dramatically reducing the development period and cost of drug delivery systems.

### BRIEF DESCRIPTION OF THE DRAWINGS

FIG. 1 shows a strategy for the development of a tumor-selective cleavable linker for antibody-drug conjugates (ADCs).

FIG. 2 shows strategies for the development and evaluation a tumor-selective cleavable linker for antibody-drug conjugates (ADCs) according to the present invention.

FIG. 3(A) to FIG. 3(C) show the chemical structures of leucine aminopeptidase (LAP)-responsive activatable fluorescent probes according to one embodiment of the present invention. FIG. 3(A) shows the design of LAP-responsive fluorescent probes, FIG. 3(B) shows the structures of fluorophores applied to activatable fluorescent probes, and FIG. 3(C) shows the structures of synthesized LAP-responsive fluorescent probes.

FIG. 4 shows pH-dependent fluorescence spectra of fluorophores (normalized fluorescence intensity plotted against pH at 1.0 μM) in buffers with a pH ranging from 2 to 13.

FIG. 5 shows thermal stability of LAP-responsive activatable fluorescent probes (1.0 μM) at various temperatures (25, 28, 31, 34, 37, 43 and 45° C.) in PBS buffer (10 mM, pH 7.4).

FIG. 6 shows pH-dependent stability of LAP-responsive activatable fluorescent probes (1.0 μM) in the pH range of 2 to 12 at 25° C. in PBS buffer (10 mM, pH 7.4).

FIG. 7(A) to FIG. 7(C) show the results of concentration optimization of LAP reaction. FIG. 7(A) shows LAP reaction of fluorescent probes (1.0 μM) on various concentrations of LAP (1, 5, 10, 20, 50, 100 g/mL), FIG. 7(B) shows LAP reaction of typical xanthene-based fluorescent probes (0.05, 0.1, 0.2, 0.5, 1 μM) on LAP (10 μg/mL), and FIG. 7(C) shows LAP reaction of reduced xanthene-based fluorescent probes (0.05, 0.1, 0.2, 0.5, 1 μM) on LAP (10 μg/mL) in PBS buffer (10 mM, pH 7.4) at 37° C. (n=3).

FIG. 8(A) and FIG. 8(B) show the results of a kinetic study of activatable fluorescent probes during leucine aminopeptidase (LAP) reactions. FIG. 8(A) shows time-dependent fluorescence emissions of typical xanthene-based fluorescent probes (1.0 μM) in the presence of 10 μg/mL of LAP in phosphate-buffered saline (PBS) buffer (100 mM, pH 7.4, and 37° C.) for 80 min, and FIG. 8(B) shows time-dependent fluorescence emissions of reduced xanthene-based fluorescent probes (1.0 μM) in the presence of 10 μg/mL of LAP in PBS buffer (100 mM, pH 7.4, and 37° C.) for 80 min.

FIG. 9(A) to FIG. 9(H) show calibration curves of fluorophores for determining the concentration of released fluorophores from activatable fluorescent probes (0.01, 0.02, 0.05, 0.1, 0.2, 0.5, 1.0, 2.0 and 5.0 μM, pH 7.4 PBS buffer). Inset: linear regression graph of fluorescence enhancement changes of concentration of fluorophore.

FIG. 10(A) and FIG. 10(B) show the results of a kinetic study of leucine aminopeptidase (LAP)-responsive fluorescent probes in HepG2 cells. FIG. 10(A) shows time-dependent fluorescence emissions of typical rhodol-based probes (1.0 μM) in HepG2 cells at 37° C. for 6 hours, and FIG. 10(B) shows the results of calculating the concentrations of fluorophores released from probes during LAP reaction.

FIG. 11(A) and FIG. 11(B) show the results of an in vitro competitive inhibition study of leucine aminopeptidase (LAP)-responsive Rhodo-P2 with LAP inhibitor, bestatin. FIG. 11(A) shows concentration-dependent inhibition of Rhodo-P2 by pretreatment with bestatin (0, 1, 10, 50, and 100 μM) in LAP (10 μg/mL) reaction (10 mM phosphate-buffered saline buffer, pH 7.4, 37° C.), and FIG. 11(B) shows concentration-dependent inhibition of Rhodo-P2 (2.0 μM) by pretreatment with bestatin (0, 10, and 100 μM) in HepG2 cells. The results are presented as mean #standard deviation (n=3).

FIG. 12(A) to FIG. 12(D) show confocal live cell imaging of leucine aminopeptidase-responsive fluorescent probes (1 μM, green) in HepG2 cells with or without pretreatment with bestatin (500 μM) for 1 hour. FIG. 12(A) shows co-localization images of Rhodo-P1 with LysoTracker™ Red DND-99 (100 nM, red) and 4′, 6-diamidino-2-phenylindole (DAPI, 10 UM, blue), FIG. 12(B) shows co-localization images of Rhodo-P2 with LysoTracker™ Red DND-99 (100 nM, red) and 4′, 6-diamidino-2-phenylindole (DAPI, 10 μM, blue), FIG. 12(C) shows co-localization images of Red Rhodo-P1 with LysoTracker™ Red DND-99 (100 nM, red) and 4′, 6-diamidino-2-phenylindole (DAPI, 10 μM, blue), and FIG. 12(D) shows co-localization images of Red Rhodo-P2 with LysoTracker™ Red DND-99 (100 nM, red) and 4′, 6-diamidino-2-phenylindole (DAPI, 10 μM, blue).

FIG. 13(A) to FIG. 13(D) show selectivity of leucine aminopeptidase (LAP)-responsive fluorescent probes (1.0 μM; Rhodo-P1 (FIG. 13(A)), Rhodo-P2 (FIG. 13(B)), Red Rhodo-P1 (FIG. 13(C)), and Red Rhodo-P2 (FIG. 13(D))) in the presence of various bioanalytes. All determinations were performed in PBS buffer (10 mM, pH 7.4) at 37° C. Bars represent relative fluorescence responses at 1 hour after the addition of bioanalytes. Normalized current response of probes in the absence and presence of 0.05 U/mL of LAP co-incubated with 1 mM of NaOCl, H₂O₂, CaCl₂), ZnCl₂, KCl, NaCl, GSH, vitamin C, L-cystein, L-arginine, L-alanine and 1 U/mL of α-amylase, lysozyme and cellulase. The results are mean±standard deviation (n=2).

FIG. 14(A) to FIG. 14(D) show ex vivo plasma stability of leucine aminopeptidase-responsive fluorescent probes. FIG. shows 14 (A) time-dependent fluorescence emissions of Rhodo-P1 and Rhodo-P2 (10 μM) in C57BL/6 mice serum for 48 hours after treatment, FIG. 14(B) shows a fluorescence calibration curve of released from LAP probes in C57BL/6 mice serum at concentrations of 0.1, 0.3, 1.0, 3.0 and 10.0 μM, FIG. 14(C) shows time-dependent survival rates of Rhodo-P1 and Rhodo-P2 in C57BL/6 mice serum for 48 hours, and FIG. 14(D) shows concentrations of probes that survived in C57BL/6 mice serum (UM) at 4, 24 and 48 hr p·i.

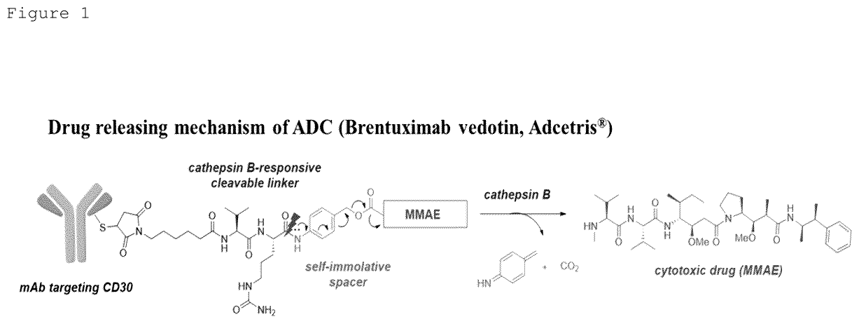

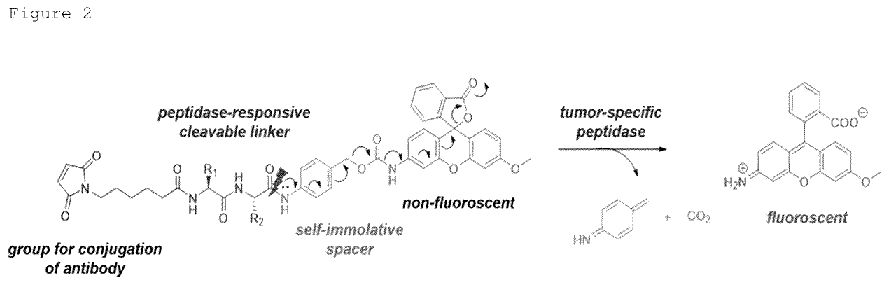

### DETAILED DESCRIPTION

Hereinafter, the present invention will be described in detail with reference to examples to aid understanding of the present invention. However, the examples according to the present invention may be modified into various other forms, and the scope of the present invention should not be construed as being limited to the following examples. The examples of the present invention are provided to explain the present invention more completely to those skilled in the art.

### Experiment Section

### 1. General Procedure for Fluorescence Analysis

All spectroscopic readings were measured with BioTek Synergy™ H1 spectrophotometer, using 96 well microplate. A parent stock solution of probes was dissolved in DMSO to obtain 10 mM. Leucine aminopeptidase was dissolved in deionized water to obtain 1 mg/mL. All spectra measurements were carried out in PBS buffer solution (PBS buffer, 10 mM, pH 7.4, 37° C. The final volume in 96 well microplate was adjusted to 200 μL. RPMI 1640 was obtained from Thermo Scientific (Waltham, MA, USA). Bestatin hydrochloride (LAP inhibitor), DMSO and 2-mercaptoethanol were purchased from Sigma Aldrich (St. Louis, Mo, USA).

2. Thermal and pH-Dependent Stability Tests

Thermal stability was evaluated by the incubation of 1 μM of the probe in PBS buffer (pH 7.4) at different temperatures ranging from 25 to 45° C. for 20 min and reading fluorescence at each temperature. The pH stability test was performed by the incubation with 1 μM probe in different pH buffers (2 to 13) at 25° C. and recording fluorescence at each pH.

3. Enzymatic Kinetic Study and Competitive Assay with LAP Inhibitor

The kinetic study for LAP reaction was performed by incubation of 2 μg of LAP (20 μg/100 μL) with 1 μM probe in PBS buffer (10 mM, pH 7.4). The plate was incubated at 37° C. for 80 min with continuous shaking, and the emission spectra were recorded at the corresponding wavelength over time. A solution of LAP (2 μg/200 μL) was pretreated with bestatin (0, 1, 10, 50 and 100 μM) in PBS (10 mM, pH 7.4) at 37° C. for 30 min, and then 1 μM of probe was added. The mixture was incubated at 37° C., and the fluorescence was recorded on Synergy™ H1 using a 96-well microplate.

### 4. Kinetic and Competitive Study in HepG2 Cells

HepG2 cells (3×10⁴ cells/well) were seeded in black-walled, transparent bottomed 96-well plates and cultured overnight. Cells were pretreated with LAP inhibitor (100 μM) for another 1 h, then cells were treated with 1 μM of probe. Kinetic study was performed in Synergy™ H1 microplate reader.

### 5. Confocal Live Cell Imaging in HepG2 Cells

HepG2 cells were maintained in DMEM medium containing 10% FBS and 1% antibiotics in the 15 μ-plate 96 well black (ibidi, Germany). HepG2 cells were incubated with or without 500 μM of bestatin, LAP inhibitor, for 1 h and subsequently treated with 1 μM of probe and Lyso-Tracker™ Red (Thermo Scientific, USA) for 1 h. The treated cells were washed three times with PBS and treated with 10 μM of DAPI (Thermo Scientific, USA) for 2 h. Imaged were collected by Leica confocal microscopy (TCS SP5 AOBS/Tandem, Leica, Germany). Lyso-Tracker™ Red was excited by 514 nm and detected by 570-620 nm, compounds were excited by 476 nm and detected by 505-545 nm, DAPI was excited by 405 nm and detected by 420-470 nm.

### 6. Selectivity Test

1 μM of probe was incubated with various bioanalytes containing 1 mM of NaOCl, H₂O₂, CaCl₂), ZnCl₂, KCl, NaCl, GSH, vitamin C, L-cystein, L-arginine, L-alanine and 1 U/mL of α-amylase, lysozyme, cellulase under 0.05 U/mL of LAP in PBS (10 mM, pH 7.4) at 37° C. for 1 h. The fluorescence was recorded on Synergy™ H1 microplate reader.

### 7. Ex Vivo Plasma Stability in Mice

Animal was treated strictly according to the guidelines for laboratory animal care and use issued by the Sunchon National University Institutional Animal Care and Use Committee (SCNU IACUC). Female C57/BL6 mice (7 weeks old), weighing about 17-20 g, were purchased from Orientbio (Orientbio Inc., Seongnam, Korea). Blood was collected from the posterior orbital plexus in normal mice and centrifuged (5000 rpm, 4° C., 5 minutes) to obtain serum. The stability study was performed by incubation of the fluorescent probe (10 μM) or rhodol fluorophore (0.1, 0.3, 1, 3 and 10 μM) with serum at 37° C. . . . The fluorescence was detected on Synergy™ H1 microplate reader.

### Experimental Results

### 1. Design, Synthesis, and Stability of Linkages

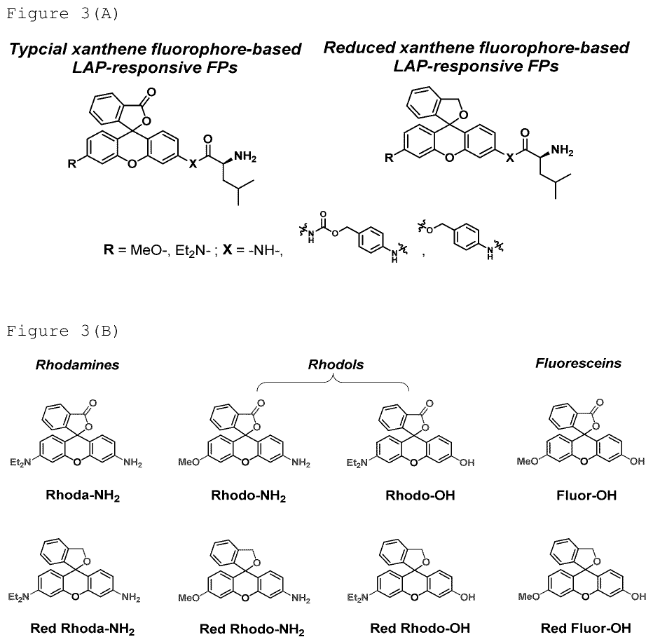

LAP is a lysosomal exopeptidase that selectively catalyzes the hydrolysis of the N-terminal leucine residue peptide and protein substrates. LAP has from numerous various biological functions and is associated with various pathological functions of cancer cells, including proliferation, invasion, angiogenesis, and drug resistance. Abnormal LAP expression is interesting as a diagnostic and prognostic biomarker of cancer. The present inventor sought to develop a new cleavable linker for application to LTD-releasing drugs, such as ADCs. Based on the biological functions of LAP and its expression levels in cancer cells, the present inventors predicted that LAP is a reasonable target for a preliminary study to identify fluorophores being efficiently released by lysosomal peptidases and investigate the effect of the p-aminobenzyl group as a spacer for linking cytotoxic drugs to cleavable linkers. Thus, the activatable fluorescent probes releasing fluorophores under the activity of LAP were designed based on our previous studies and synthesized using typical and reduced xanthene fluorophores, including fluorescein, rhodol, and rhodamine derivatives with a terminal —OH or —NH₂ group (FIG. 3(A)-(D)). The leucine residue for recognition by LAP was directly conjugated to four fluorophores with a terminal NH₂ group (Rhoda-P1, Red Rhoda-P1, Rhodo-P1, Red Rhodo-P1, FIG. 3(C)) and also linked to eight fluorophores with an —NH₂ (Rhoda-P2, Red Rhoda-P2, Rhodo-P2, Red Rhodo-P2, FIG. 3(C)) or —OH (Rhodo-P3, Red Rhodo-P3, Fluor-P3, Red Fluor-P3, FIG. 3(C)) group via a p-amino benzyl spacer using an ether or carbamate linkage.

Typical xanthene and reduced xanthene fluorophores with a terminal —NH₂ group for activatable fluorescent probes were synthesized from fluorescein as described previously, except for the typical rhodamine fluorophore (Scheme S1) [23,24]. Four activatable fluorescent probes, including Rhoda-P1, Rhodo-P1, Red Rhoda-P1, and Red Rhodo-P1, directly conjugated with a leucine, were prepared via an EEDQ coupling reaction of the corresponding fluorophores (Rhoda-NH₂, Rhodo-NH₂, Red Rhoda-NH₂, and Red Rhodo-NH₂) with Boc-Leu-OH followed by N-Boc deprotection using trifluoroacetic acid (TFA) (Scheme S2). The synthesis of four activatable fluorescent probes with a p-aminobenzyl spacer via carbamate linkages (Rhoda-P2, Rhodo-P2, Red Rhoda-P2, and Red Rhodo-P2) was initiated by the activation of fluorophores using phenyl chloroformate to produce phenoxy carbonyl intermediates. The substitution of the phenoxy group with a p-aminobenzyl alcohol coupled with Boc-Leu-OH and subsequent N-Boc deprotection using TFA then yielded the activatable fluorescent probes linked to a spacer via carbamate linkages (Scheme S3). Four fluorophores with a terminal-OH group (Rhodo-OH, Fluor-OH, and their reduced forms) were used to generate activatable fluorescent probes linked to a spacer via ether linkages (Rhodo-P3, Red Rhodo-P3, Fluor-P3, and Red Fluor-P3). O-Alkylation of the phenol group in fluorophores under Ag₂O was performed with p-aminobenzyl bromides coupled with Boc-Leu-OH, and N-Boc the deprotection then yielded corresponding final products with ether linkages (Scheme S4).

The specific synthesis method is as follows.

### A. Synthesis Materials

All reagents and solvents were obtained from commercial sources Sigma-Aldrich Chemical Co., Combi-Block, Tokyo Chemical Industries, Daejung Chemicals, and Alfa Aesar and used without purification. Anhydrous solvents were purchased from Sigma-Aldrich and used under dry nitrogen atmosphere. Ultrapure water was obtained from a water ultra-purification system. Completion of reaction was confirmed by thin layer chromatography (TLC) on Kiesegel 60F₂₅₄ from Merck and all synthesized compounds were purified by flash column chromatography using ZEOprep 60 (40-63) μM silica gel from ZEOCHEM. ¹H and ¹³C NMR spectra were measured on JEOL JNMECZ400s/L1 (400 MHZ) and CDCl₃ or DMSO-d₆ was used as the NMR solvents. The chemical shifts were quoted in parts per million (ppm) and the coupling constant (J) were reported in hertz unit (Hz). Chemical shifts (in ppm) were referenced to tetramethylsilane (δ=0 ppm) in CDCl3 as an internal standard. ¹³C NMR spectra were reported in ppm referenced to the center line of the triplet at 77.0 ppm for CDCl₃ or 39.5 ppm for DMSO-d₆. All in vitro enzyme assays were performed by taking the absorbance and emission on Synergy™ H1 microplate reader from BioTek. Leucine aminopeptidase from porcine kidney and other enzymes were purchased from Sigma-Aldrich.

### B. Synthesis Method

### B-1. General Procedure A for Amide Coupling

To a solution of fluorophore (1.2 eq.) in CH₂Cl₂ was added EEDQ (1.2 eq.) and Boc-Leu-OH (1.0 eq.) The reaction mixture was stirred at RT for 4 h under N₂ atmosphere. After completion of reaction, the solvent was removed under reduced pressure and the residue was purified by flash column chromatography on silica gel to give the desired probe with a amide linkage.

### B-2. General Procedure B for the Carbamate Linkage

To a solution of fluorophore with a terminal —NH₂ group (1.0 eq) in CH₂Cl₂ was added a stock solution of phenyl chloroformate (1 M in CH₂Cl₂) and DIPEA (1 M in CH₂Cl₂) at 0° C. The reaction mixture was stirred at RT for 2 h. The reaction was quenched with water and extracted with CH₂Cl₂. The organic layer was washed with water and dried over Na₂SO₄. The organic layer was filtered and concentrated under reduced pressure. The crude residue was purified by flash column chromatography on silica gel to afford the desired phenyl carbamate. The obtained phenyl carbamate (1.00 eq) and DBU (1.00 eq.) was added to a solute on of the benzyl alcohol 41 (1.00 eq.) in acetone. The mixture was stirred at RT for 1 h and then the solvent was removed under reduced pressure. The crude residue was purified by flash column chromatography on silica gel to produce the desired probe with a carbamate linkage.

### B-3. General Procedure C for the Ether Linkage

To a solution of fluorophore with a terminal-OH group (1.0 eq.) in toluene was added the benzyl bromide 42 (1.2 eq.) and Ag₂O (1.5 eq.). The reaction mixture was heated at 120° C. for 2 to 12 h under N₂ atmosphere. After completion of reaction, the solvent was removed under reduced eq.) in acetone. The mixture was stirred at RT for 1 h and then the solvent was removed under reduced pressure and the crude residue was purified by flash column chromatography on silica gel to give the desired probe with an ether linkage.

### B-4. General Procedure D for the N-Boc-Deprotection

To a solution of N-Boc-protected probe (10 mg) in anhydrous CH₂Cl₂ (1 mL) was added dropwise a 30% solution of TFA in CH₂Cl₂ (0.6 mL) at −20° C. and was stirred overnight at −20° C. The saturated aqueous NaHCO₃ solution was added to the reaction until pH 8 and the mixture was extracted with CH₂Cl₂. The combined organic layers were washed with water and dried over Na₂SO₄. The crude residue was purified by flash column chromatography on silica gel to produce the desired product.

### C. Synthesis Reaction

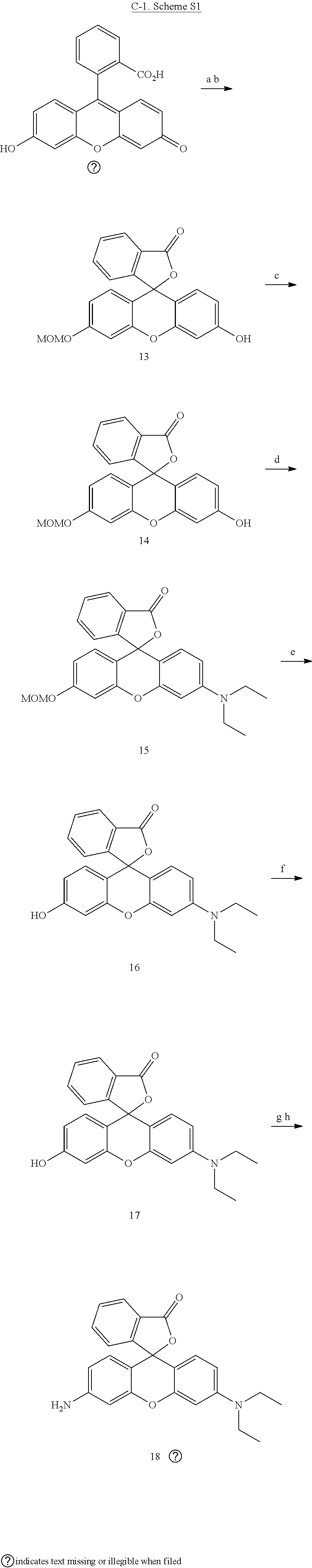

Synthesis of rhodamine fluorophore 18. Reagents and conditions: (a) MOMCl, K₂CO₃·DMF, rt; (b) NaOH, THE/WATER, reflux, 13% for 2 steps; (c) Tf₂O, pyridine, CH₂Cl₂, 0° C. to rt, 82%; (d) Diethylamine, Pd(OAc)₂, CS₂CO₃, BINAP, toluene, reflux, 76%; (e) TFA, CH₂Cl₂, rt, 90%; (f) N-Phenyl-bis-(trifluoromethanesulfonimide), K₂CO₃, CH₃CN, 0° C. to rt, 49%; (g) Benzophenone Imine, Pd(OAc)₂, Cs₂CO₃, BINAP, toluene, reflux; (h) aq. 1N—HCl, THF, rt, 89% for 2 steps

### 3′-Hydroxy-6′-(methoxymethoxy)-3H-spiro[isobenzofuran-1,9′-xanthen]-3-one (13)

To a solution of fluorescein (10 g, 30 mmol) and K₂CO₃ (10.4 g, 75 mmol) in DMF (150 mL) was added chloromethyl methyl ether (7.7 g, 90 mmol) at RT for 12 h under nitrogen atmosphere. The mixture was extracted with EtOAc and washed with 1N HCl solution, water and brine. The organic layer was dried with Na₂SO₄ and concentrated under reduced pressure to provide a solid. The obtained solid was dissolved in a mixture of THF (150 mL) and aqueous NaOH solution (3 M, 0 mL). The mixture was heated to reflux for 2 h and cooled to RT. The residue was acidified with aqueous 1N HCl to pH 2 and then extracted with EtOAc. The organic layer was washed with water and brine, dried over Na₂SO₄ and concentrated under reduced pressure. The crude residue was purified by column chromatography on silica gel (EtOAc:Hexane=1:2) to afford the compound 13 (1.47 g, 13%).

¹H NMR (400 MHZ, DMSO-d₆) δ 8.05 (d, J=6.9 Hz, 1H), 7.75-7.83 (m, 2H), 7.71 (d, J=2.3 Hz, 1H), 7.39 (d, J=7.8 Hz, 1H), 7.25 (dd, J=8.9, 2.5 Hz, 1H), 7.06 (d, J=1.8 Hz, 2H), 6.84 (dd, J=8.9, 2.5 Hz, 1H), 6.74-6.77 (m, 1H), 6.62 (s, 1H), 5.27 (d, J=1.8 Hz, 2H), 3.38 (s, 3H); ¹³C NMR (100 MHz, DMSO-d₆) δ 168.36, 158.66, 152.05, 151.24, 150.99, 149.67, 136.05, 130.59, 129.18, 125.41, 125.05, 124.96, 124.15, 119.76, 117.42, 113.97, 111.42, 110.75, 103.30, 102.29, 93.94, 80.81, 55.83; HRMS (ESI⁺): m/z Calcd for C₂₂H₁₆O₆ [M]⁺: 376.0947, Found: 376.0949.

### 3′-(Methoxymethoxy)-3-oxo-3H-spiro[isobenzofuran-1,9′-xanthen]-6′-yl-trifluoromethanesulfonate (14)

To a solution of compound 13 (100 mg, 0.266 mmol) and pyridine (85 mg, 1.064 mmol) in anhydrous CH₂Cl₂ (5 mL) was added dropwise trifluoromethanesulfonic anhydride (153 mg, 0.532 mmol) at 0° C. under nitrogen atmosphere. The mixture was stirred at RT for 3 h under nitrogen atmosphere and then was quenched with water. The residue was extracted with CH₂Cl₂ and washed with 1N HCl, water and brine. The organic layer was dried with Na₂SO₄ and concentrated. The product was isolated by flash column chromatography on silica gel (EtOAc:Hexane=1:2) to yield the compound 14 (110 mg, 82%).

¹H NMR (400 MHZ, CDCl₃) δ 8.05 (d, J=7.8 Hz, 1H), 7.68 (dtd, J=19.4, 7.4, 1.1 Hz, 2H), 7.25 (d, J=2.3 Hz, 1H), 7.18 (d, J=7.3 Hz, 1H), 7.00 (d, J=2.3 Hz, 1H), 6.96 (dd, J=8.9, 2.5 Hz, 1H), 6.90 (d, J=8.7 Hz, 1H), 6.78 (dd, J=9.1, 2.3 Hz, 1H), 6.73 (d, J=8.7 Hz, 1H), 5.21 (s, 2H), 3.48 (s, 3H); ¹³C NMR (100 MHZ, CDCl₃) δ 168.8, 159.1, 152.2, 151.9, 149.8, 135.2, 130.0, 129.7, 128.8, 126.1, 125.1, 123.8, 119.7, 118.5, 116.4, 113.8, 111.6, 110.1, 103.5, 94.2, 81.4, 55.8; HRMS (ESI⁺): m/z Calcd for C₂₃H₁₅F₃O₆S [M]⁺: 508.0440, Found: 508.0438.

### 3′-(Diethylamino)-6′-(methoxymethoxy)-3H-spiro[isobenzofuran-1,9′-xanthen]-3-one (15)

An oven-dried round bottom flask was charged with Pd(OAc)₂ (59 mg, 0.256 mmol), Cs₂CO₃ (1.18 g, 3.584 mmol) and BINAP (244 mg, 0.384 mmol) and was flushed with nitrogen gas for 10 min. A solution of compound 14 (1.3 g, 2.56 mmol) and diethylamine (1.91 g, 25.6 mmol) in anhydrous toluene (83 mL) was added, stirred under nitrogen atmosphere at RT for 20 min and heated at 100° C. for 20 h. After completion of the reaction, the reaction mixture was cool down to RT, filtered through a pad of Celite and washed with CH₂Cl₂. The filtrate was concentrated and purified by flash column chromatography on silica gel (EtOAc:Hexane=1:2) to afford the compound 15 (1.1 g, 76%).

¹H NMR (400 MHZ, DMSO-d₆) δ 7.98 (d, J=7.8 Hz, 1H), 7.78 (dd, J=7.3, 6.4 Hz, 1H), 7.71 (t, J=7.3 Hz, 1H), 7.27 (d, J=7.8 Hz, 1H), 6.96 (d, J=2.3 Hz, 1H), 6.75 (dd, J=8.9, 2.5 Hz, 1H), 6.64 (d, J=8.7 Hz, 1H), 6.45 (s, 3H), 5.24 (s, 2H), 3.41 (m, 4H), 3.37 (s, 3H), 1.08 (t, J=6.9 Hz, 6H); ¹³C NMR (100 MHZ, DMSO-d₆) δ 168.78, 158.30, 152.38, 152.22, 149.26, 135.57, 130.10, 129.05, 128.68, 124.60, 124.10, 112.77, 112.52, 108.70, 103.26, 96.88, 93.92, 83.43, 55.80, 54.94, 43.81, 12.33; HRMS (ESI⁺): m/z Calcd for C₂₆H₂₅NO₅ [M]⁺: 431.1733, Found: 431.1738.

### 3′-(Diethylamino)-6′-hydroxy-3H-spiro[isobenzofuran-1,9′-xanthen]-3-one (16)

To a solution of compound 15 (100 mg, 0.23 mmol) in anhydrous CH₂Cl₂ (1 mL) was added dropwise the solution of trifluoroacetic acid (1 mL) at 0° C. The reaction mixture was stirred at 0° C. for 30 min and then warmed to RT followed by stirring of 1 h. After completion of reaction, the mixture was quenched with aqueous NaHCO₃ to pH 7 and extracted with EtOAc, washed with water. The organic layer was dried with Na₂SO₄. The crude residue was purified by flash column chromatography on silica gel (CH₂Cl₂:MeOH=5:1) to produce 16 (80 mg, 90%).

¹H NMR (400 MHZ, CDCl3) δ 8.09 (d, J=8.7 Hz, 1H), 7.60-7.53 (m, 2H), 7.11 (d, J=8.7 Hz, 1H), 6.93-6.83 (m, 2H), 6.74 (d, J=2.3 Hz, 1H), 6.62-6.53 (m, 3H); ¹³C NMR (100 MHZ, CDCl3) δ 167.3, 166.5, 161.9, 159.0, 157.5, 156.7, 133.7, 132.5, 132.1, 131.2, 131.1, 130.9, 130.2, 130.1, 117.0, 116.5, 115.6, 115.2, 102.0, 96.0, 46.2, 11.6; HRMS (ESI⁺): m/z Calcd for C₂₄H₂₁NO₄ [M]⁺: 387.1471, Found: 387.1472.

### 3′-(Diethylamino)-3-oxo-3H-spiro[isobenzofuran-1,9′-xanthen]-6′-yl-trifluoromethanesulfonate (17)

To a solution of compound 16 (200 mg, 0.516 mmol) in MeCN (20 mL) was added phenyl triflimide (372 mg, 1.032 mmol) and K₂CO₃ (287 mg, 2.064 mmol). The reaction mixture was stirred at RT for 12 h. After completion of the reaction, the solvent was removed under reduced pressure and the residue was extracted with CH₂Cl₂ and water. The organic layer was washed with brine and dried over Na₂SO₄. The crude residue was purified by flash column chromatography on silica gel (EtOAc:Hexane=1:8) to produce 17 (130 mg, 49%).

¹H NMR (400 MHZ, CDCl₃) δ 8.03 (d, J=7.8 Hz, 1H), 7.73-7.61 (m, 2H), 7.23-7.17 (m, 2H), 6.92 (dd, J=8.7, 2.3 Hz, 1H), 6.87 (d, J=8.7 Hz, 1H), 6.58 (d, J=48.5 Hz, 3H), 3.38 (d, J=6.9 Hz, 4H), 1.19 (t, J=7.1 Hz, 6H); ¹³C NMR (100 MHZ, CDCl₃) δ 169.16, 152.60, 149.89, 135.06, 130.97, 130.01, 128.80, 126.92, 125.12, 124.00, 120.28, 119.78, 117.65, 116.11, 110.31, 109.07, 104.42, 97.59, 82.79, 44.53, 12.45; HRMS (ESI⁺): m/z Calcd for C₂₅H₂₀FNO₆S [M+H]⁺: 519.0963, Found 520.1035.

### 3′-Amino-6′-(diethylamino)-3H-spiro[isobenzofuran-1,9′-xanthen]-3-one (18, Rhoda-NH₂)

An oven-dried round bottom flask was charged with Pd(OAc)₂ (16 mg, 0.023 mmol), Cs₂CO₃ (227 mg, 0.69 mmol) and BINAP (24 mg, 0.037 mmol) and flushed with nitrogen gas for 10 min. A solution of compound 17 (120 mg, 0.23 mmol) and benzophenone imine (53 mg, 0.276 mmol) in anhydrous toluene (6 mL) was added and stirred under nitrogen atmosphere at RT for 20 min and then heated at 100° C. for 4 h. The mixture was filtered through a pad of Celite and washed with CH₂Cl₂. The filtrate was concentrated and re-dissolved in THF (2.4 mL). The mixture was added dropwise 1N HCl (0.8 mL). The reaction mixture was stirred to RT for 1 h. The reaction was neutralized with saturated aqueous NaHCO₃ to pH 7-8. The residue was extracted with EtOAc and washed with water. The organic layer was washed with water and brine, dried over Na₂SO₄ and concentrated under reduced pressure. The crude residue was purified by column chromatography on silica gel (CH₂Cl₂:MeOH=10:1) to afford 18 (Rhoda-NH2, 78 mg, 89%).

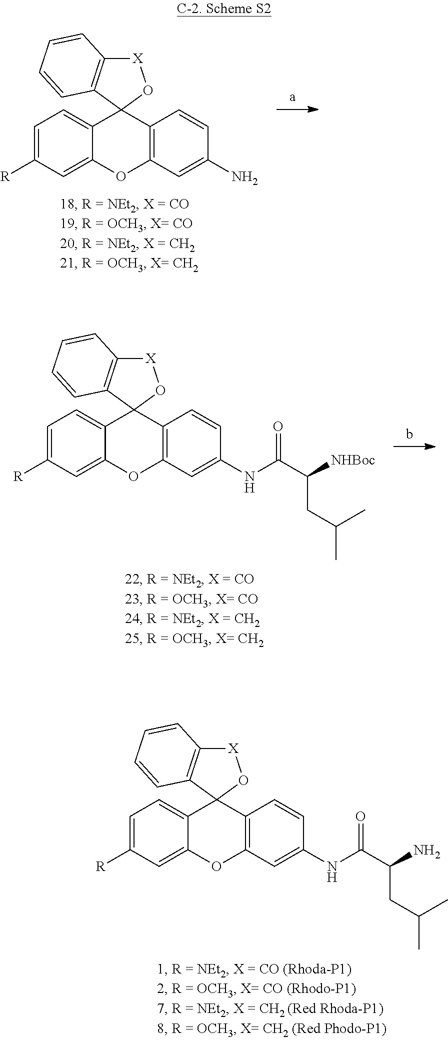

Synthesis of LAP-responsive activatable fluorescent probes via direct amide linkage. Reagents and conditions: (a) Boc-Leu-OH, EEDQ, CH₂Cl₂, rt, 22=92%, 23=67%, 24=56%, 25=81%; (b) 30% TFA in CH₂Cl₂, −20° C., 1=30%, 2=80%, 7=52%, 8=29%.

### tert-Butyl ((2S)-1-((3′-(diethylamino)-3-oxo-3H-spiro[isobenzofuran-1,9′-xanthen]-6′-yl)amino)-4-methyl-1-oxopentan-2-yl)carbamate (22)

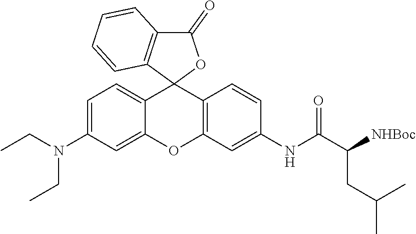 [SMILES: CCN(CC)c1ccc2c(c1)Oc1cc(NC(=O)[C@H](CC(C)C)NC(=O)OC(C)(C)C)ccc1C21OC(=O)c2ccccc21]

Compound 22 was synthesized by the amide coupling of 18 (Rhoda-NH2, 30 mg, 0.078 mmol) with Boc-Leu-OH (15 mg, 0.065 mmol) and EEDQ (19 mg, 0.078 mmol) according to the general procedure A. The crude residue was purified by flash column chromatography on silica gel (CH₂Cl₂:MeOH=30:1) to produce 22 in 92% of yield (37 mg).

¹H NMR (400 MHZ, DMSO-d₆) δ 10.24 (s, 1H), 7.98 (d, J=7.8 Hz, 1H), 7.85 (s, 1H), 7.78 (t, J=7.3 Hz, 1H), 7.70 (t, J=7.5 Hz, 1H), 7.26 (d, J=7.8 Hz, 1H), 7.11 (dd, J=13.7, 8.2 Hz, 2H), 6.68 (d, J=8.7 Hz, 1H), 6.47 (d, J=14.2 Hz, 3H), 4.11 (d, J=5.0 Hz, 1H), 3.36-3.32 (m, 4H), 1.63-1.51 (m, 2H), 1.43-1.30 (m, 1H), 1.37 (s, 9H), 1.08 (t, J=6.9 Hz, 6H), 0.88 (t, J=4.8 Hz, 6H); ¹³C NMR (100 MHz, DMSO-d₆) δ 172.5, 168.8, 155.5, 152.5, 152.2, 151.3, 149.3, 141.0, 135.5, 130.0, 128.6, 128.4, 126.3, 124.6, 124.0, 114.9, 113.6, 108.6, 106.3, 104.5, 97.0, 83.3, 78.1, 53.6, 43.8, 28.2, 24.3, 22.9, 21.5, 12.3; HRMS (ESI⁺): m/z Calcd for C₃₅H₄₁N₃O₆ [M+H]⁺: 600.3029, Found: 600.3069.

### tert-Butyl ((2S)-1-((3′-methoxy-3-oxo-3H-spiro[isobenzofuran-1,9′-xanthen]-6′-yl)amino)-4-methyl-1-oxopentan-2-yl)carbamate (23)

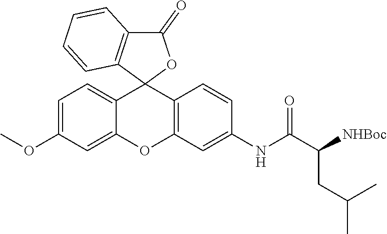 [SMILES: COc1ccc2c(c1)Oc1cc(NC(=O)[C@H](CC(C)C)NC(=O)OC(C)(C)C)ccc1C21OC(=O)c2ccccc21]

Compound 23 was synthesized by the amide coupling of 19 (Rhodo-NH₂, 90 mg, 0.259 mmol) with Boc-Leu-OH (51 mg, 0.216 mmol) and EEDQ (65 mg, 0.259 mmol) according to the general procedure A. The crude residue was purified by flash column chromatography on silica gel (EtOAc:Hexane=1:4) to produce 23 in 67% of yield (80 mg).

¹H NMR (400 MHZ, DMSO-d₆) δ 10.28 (s, 1H), 8.01 (d, J=7.8 Hz, 1H), 7.89 (d, J=3.7 Hz, 1H), 7.79 (t, J=7.1 Hz, 1H), 7.72 (t, J=7.5 Hz, 1H), 7.26 (d, J=7.1 Hz, 1H), 7.18 (d, J=8.2 Hz, 1H), 7.10 (d, J=8.2 Hz, 1H), 6.97 (d, J=2.3 Hz, 1H), 6.78-6.63 (m, 3H), 4.14-4.01 (m, 1H), 3.81 (s, 3H), 1.62-1.65 (m, 1H), 1.58-1.46 (m, 1H), 1.37 (s, 9H), 1.3 (s, 1H), 0.92-0.82 (m, 6H); ¹³C NMR (100 MHz, DMSO-d₆) δ 172.54, 168.68, 161.10, 155.51, 152.57, 151.79, 150.82, 141.16, 135.76, 130.24, 128.95, 128.48, 125.76, 124.76, 123.97, 115.40, 113.15, 112.13, 110.72, 106.24, 100.85, 82.13, 78.08, 55.69, 53.65, 28.19, 24.34, 22.93, 21.52; HRMS (ESI⁺): m/z Calcd for C₃₂H₃₄N₂O₇ [M+H]⁺: 559.2400, Found: 559.2447.

### tert-Butyl ((25)-1-((3′-(diethylamino)-3H-spiro[isobenzofuran-1,9′-xanthen]-6′-yl)amino)-4-methyl-1-oxopentan-2-yl)carbamate (24)

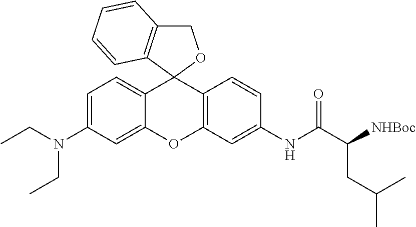 [SMILES: CCN(CC)c1ccc2c(c1)Oc1cc(NC(=O)[C@H](CC(C)C)NC(=O)OC(C)(C)C)ccc1C21OCc2ccccc21]

Compound 24 was synthesized by the amide coupling of 20 (Red Rhoda-NH₂, 50 mg, 0.134 mmol) with Boc-Leu-OH (26 mg, 0.112 mmol) and EEDQ (34 mg, 0.134 mmol) according to the general procedure A. The crude residue was purified by flash column chromatography on silica gel (EtOAc:Hexane=1:4) to produce 24 in 56% of yield (34 mg).

¹H NMR (400 MHZ, DMSO-d₆) δ 7.73 (q, J=1.8 Hz, 1H), 7.43 (d, J=7.3 Hz, 1H), 7.35 (t, J=7.3 Hz, 1H), 7.23 (t, J=7.3 Hz, 1H), 7.13 (dq, J=8.6, 1.9 Hz, 1H), 6.81 (d, J=8.2 Hz, 1H), 6.76 (d, J=7.8 Hz, 1H), 6.66 (d, J=8.7 Hz, 1H), 6.40 (td, J=8.1, 2.4 Hz, 2H), 5.21 (s, 2H), 3.34 (d, J=7.3 Hz, 5H), 2.07 (s, 2H), 1.79-1.69 (m, 1H), 1.50-1.43 (m, 1H), 1.36-1.29 (m, 1H), 1.08 (t, J=6.9 Hz, 6H), 0.88 (dd, J=10.1, 6.9 Hz, 6H); ¹³C NMR (100 MHZ, DMSO-d₆) δ 175.2, 151.0, 150.0, 148.2, 145.3, 139.4, 138.7, 129.4, 128.9, 128.1, 127.8, 123.1, 121.0, 120.0, 114.4, 111.4, 108.1, 105.8, 96.8, 82.7, 71.4, 54.0, 44.0, 43.6, 30.6, 24.2, 23.1, 21.8, 12.4; HRMS (ESI⁺): m/z Calcd for C₃₅H₄₃N₃O₅ [M+H]⁺: 586.3236, Found: 586.3278.

### tert-Butyl((2S)-1-((3′-methoxy-3H-spiro[isobenzofuran-1,9′-xanthen]-6′-yl)amino)-4-methyl-1-oxopentan-2-yl)carbamate (25)

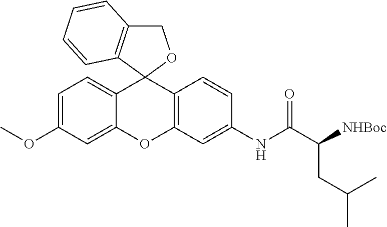 [SMILES: COc1ccc2c(c1)Oc1cc(NC(=O)[C@H](CC(C)C)NC(=O)OC(C)(C)C)ccc1C21OCc2ccccc21]

Compound 25 was synthesized by the amide coupling of 21 (Red Rhodo-NH₂, 50 mg, 0.151 mmol) with Boc-Leu-OH (30 mg, 0.126 mmol) and EEDQ (38 mg, 0.151 mmol) according to the general procedure A. The crude residue was purified by flash column chromatography on silica gel (EtOAc:Hexane=1:3) to produce 25 in 81% of yield (56 mg).

¹H-NMR (400 MHZ, DMSO-d₆) δ 10.09 (d, J=11.0 Hz, 1H), 7.74-7.72 (m, 1H), 7.45 (d, J=7.3 Hz, 1H), 7.36 (t, J=3.9 Hz, 1H), 7.23 (t, J=4.1 Hz, 1H), 7.12 (d, J=11.4 Hz, 1H), 6.90-6.81 (m, 3H), 6.74 (d, J=7.8 Hz, 1H), 6.66 (dd, J=8.7, 2.7 Hz, 1H), 5.28 (s, 2H), 4.18-4.06 (m, 1H), 3.77 (s, 3H), 1.68-1.59 (m, 1H), 1.55-1.47 (m, 1H), 1.44-1.39 (m, 1H), 1.36 (s, 9H), 0.91-0.86 (m, 6H); ¹³C-NMR (100 MHZ, DMSO-d₆) δ 172.3, 154.6, 150.8, 148.8, 144.5, 139.5, 138.1, 131.5, 128.9, 128.1, 126.7, 124.6, 121.2, 120.2, 113.7, 111.6, 98.7, 95.6, 94.5, 79.1, 66.9, 62.2, 48.6, 41.4, 32.8, 30.5, 28.2, 22.8, 21.6; HRMS (ESI⁺): m/z Calcd for C₃₂H₃₆N₂O₆ [M+H]⁺: 545.2607, Found: 545.2659.

### (2S)-2-Amino-N-(3′-(diethylamino)-3-oxo-3H-spiro[isobenzofuran-1,9′-xanthen]-6′-yl)-4-methylpentanamide (1, Rhoda-P1)

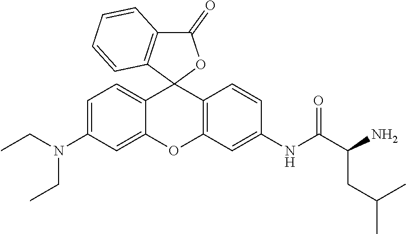 [SMILES: CCN(CC)c1ccc2c(c1)Oc1cc(NC(=O)[C@@H](N)CC(C)C)ccc1C21OC(=O)c2ccccc21]

Probe 1 (Rhoda-P1) was synthesized from 22 (35 mg, 0.078 mmol) according to the general procedure D. The residue was purified by flash column chromatography on silica gel (CH2Cl2:MeOH=10:1) to give probe 1 (Rhoda-P1) in 30% of yield (9 mg).

¹H NMR (400 MHz, DMSOd6) δ 7.98 (d, J=7.3 Hz, 1H), 7.88 (s, 1H), 7.78 (t, J=7.5 Hz, 1H), 7.70 (t, J=7.3 Hz, 1H), 7.26 (d, J=7.8 Hz, 1H), 7.18 (dd, J=8.5, 1.6 Hz, 1H), 6.66 (d, J=8.7 Hz, 1H), 6.47 (d, J=12.8 Hz, 3H), 3.37-3.32 (m, 5H), 1.79-1.69 (m, 1H), 1.49-1.42 (m, 1H), 1.36-1.29 (m, 1H), 1.08 (t, J=6.9 Hz, 6H), 0.88 (dd, J=10.3, 6.6 Hz, 6H); ¹³C NMR (100 MHZ, DMSO-d₆) δ 176.0, 169.2, 152.9, 152.7, 151.7, 149.7, 141.4, 136.0, 130.4, 129.0, 128.7, 126.7, 125.0, 124.5, 115.4, 114.0, 109.0, 106.6, 104.9, 97.4, 83.8, 54.6, 44.4, 44.2, 24.6, 23.6, 22.3, 12.8; HRMS (ESI⁺): m/z Calcd for C₃₀H₃₃N₃O₄ [M+H]⁺: 500.2505, Found: 500.2542.

### (2S)-2-Amino-N-(3′-methoxy-3-oxo-3H-spiro[isobenzofuran-1,9′-xanthen]-6′-yl)-4-methylpentanamide (2, Rhodo-P1)

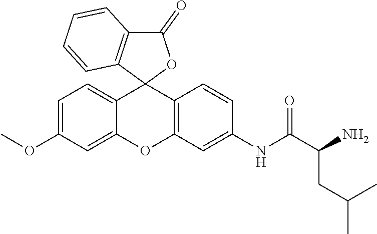 [SMILES: COc1ccc2c(c1)Oc1cc(NC(=O)[C@@H](N)CC(C)C)ccc1C21OC(=O)c2ccccc21]

Probe 2 (Rhodo-P1) was synthesized from 23 (30 mg, 0.54 mmol) according to the general procedure D, using 30% trifluoroacetic acid in CH₂Cl₂ (1.8 mL). The residue was purified by flash column chromatography on silica gel (CH₂Cl₂:MeOH=10:1) to give 20 mg of probe 2 (Rhodo-P1) in 80% of yield.

¹H NMR (400 MHZ, DMSO-d₆) δ 8.01 (d, J=7.3 Hz, 1H), 7.94 (d, J=8.2 Hz, 1H), 7.79 (t, J=7.5 Hz, 1H), 7.72 (t, J=7.1 Hz, 1H), 7.31-7.18 (m, 2H), 6.97 (d, J=2.3 Hz, 1H), 6.77-6.64 (m, 3H), 3.81 (s, 3H), 3.38 (s, 1H) 3.35 (s, 2H), 1.80-1.67 (m, 1H), 1.52-1.42 (m, 1H), 1.40-1.29 (m, 1H), 0.88 (dd, J=9.6, 6.9 Hz, 6H); ¹³C NMR (100 MHZ, DMSO-d₆) δ 175.31, 168.66, 161.10, 152.53, 151.80, 150.83, 141.09, 135.74, 130.23, 128.95, 125.78, 124.76, 123.96, 115.44, 113.10, 112.10, 110.73, 106.18, 100.83, 82.16, 55.68, 54.04, 43.78, 24.18, 23.14, 21.84; HRMS (ESI⁺): m/z Calcd for C₂₇H₂₆N₂O₅ [M+H]⁺: 459.1875, Found: 459.1917.

### (2S)-2-Amino-N-(3′-(diethylamino)-3H-spiro[isobenzofuran-1,9′-xanthen]-6′-yl)-4-methylpentanamide (7, Red Rhoda-P1)

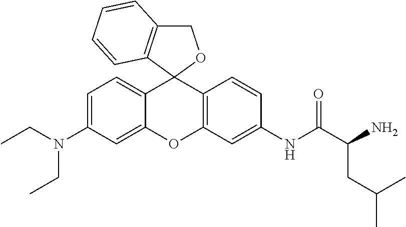 [SMILES: CCN(CC)c1ccc2c(c1)Oc1cc(NC(=O)[C@@H](N)CC(C)C)ccc1C21OCc2ccccc21]

Probe 7 (Red Rhoda-P1) was synthesized of compound 24 (30 mg, 0.055 mmol) according to the general procedure D. The residue was purified by flash column chromatography on silica gel (CH₂Cl₂:EA=5:1) to give probe 7 (Red Rhoda-P1) in 52% of yield (14 mg).

¹H NMR (400 MHZ, DMSO-d₆) δ 7.74 (q, J=2.1 Hz, 1H), 7.44 (d, J=7.3 Hz, 1H), 7.35 (t, J=7.3 Hz, 1H), 7.23 (t, J=7.3 Hz, 1H), 7.13 (qd, J=4.2, 2.2 Hz, 1H), 6.81 (d, J=8.7 Hz, 1H), 6.76 (d, J=7.3 Hz, 1H), 6.66 (d, J=8.7 Hz, 1H), 6.40 (td, J=8.7, 2.3 Hz, 2H), 5.21 (s, 2H), 3.35 (m, 5H), 2.53 (s, 2H), 1.79-1.69 (m, 1H), 1.49-1.42 (m, 1H), 1.35-1.28 (m, 1H), 1.08 (t, J=6.9 Hz, 6H), 0.88 (dd, J=10.1, 6.9 Hz, 6H); ¹³C NMR (100 MHZ, DMSO-d₆) δ 175.3, 151.0, 150.0, 148.2, 145.4, 139.5, 138.7, 129.5, 129.0, 128.1, 127.8, 123.2, 121.1, 120.0, 114.4, 111.3, 108.1, 105.8, 96.8, 82.7, 71.4, 54.1, 44.0, 43.7, 40.4, 24.2, 23.2, 21.9, 12.4; HRMS (ESI⁺): m/z Calcd for C₃₀H₃₅N₃O₃ [M+H]⁺: 486.2712, Found: 486.2750.

### (2S)-2-Amino-N-(3′-methoxy-3H-spiro[isobenzofuran-1,9′-xanthen]-6′-yl)-4-methylpentanamide (8, Red Rhodo-P1)

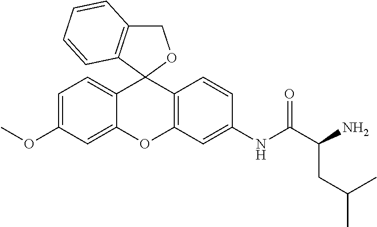 [SMILES: COc1ccc2c(c1)Oc1cc(NC(=O)[C@@H](N)CC(C)C)ccc1C21OCc2ccccc21]

Probe 8 (Red Rhodo-P1) was synthesized from compound 25 (20 mg, 0.37 mmol) according to the general procedure D, using 30% trifluoroacetic acid in CH₂Cl₂ (1.2 mL). The residue was purified by flash column chromatography on silica gel (CH₂Cl₂:MeOH=10:1) to give 5 mg of probe 8 (Red Rhodo-P1) in 29% of yield.

¹H-NMR (400 MHZ, DMSO-d₆) δ 7.75 (q, J=1.8 Hz, 1H), 7.45 (d, J=7.3 Hz, 1H), 7.35 (t, J=7.1 Hz, 1H), 7.23 (t, J=7.1 Hz, 1H), 7.16 (dq, J=8.5, 1.8 Hz, 1H), 6.89-6.80 (m, 3H), 6.73 (d, J=7.3 Hz, 1H), 6.66 (dd, J=8.9, 2.5 Hz, 1H), 5.28 (s, 2H), 3.77 (s, 3H), 2.07 (s, 2H), 1.81-1.69 (m, 1H), 1.51-1.43 (m, 1H), 1.36-1.29 (m, 1H), 0.93-0.85 (m, 6H); ¹³C-NMR (100 MHZ, DMSO-d₆) δ 175.3, 159.9, 150.5, 149.6, 145.3, 139.6, 138.3, 129.6, 128.9, 128.2, 128.0, 123.0, 121.2, 119.6, 117.2, 114.9, 111.2, 105.8, 82.4, 71.9, 55.4, 55.3, 54.1, 44.0, 30.6, 24.2, 23.1, 21.8; HRMS (ESI⁺): m/z Calcd for C₂₇H₂₈N₂O₄ [M+H]⁺: 445.2083, Found: 445.2128.

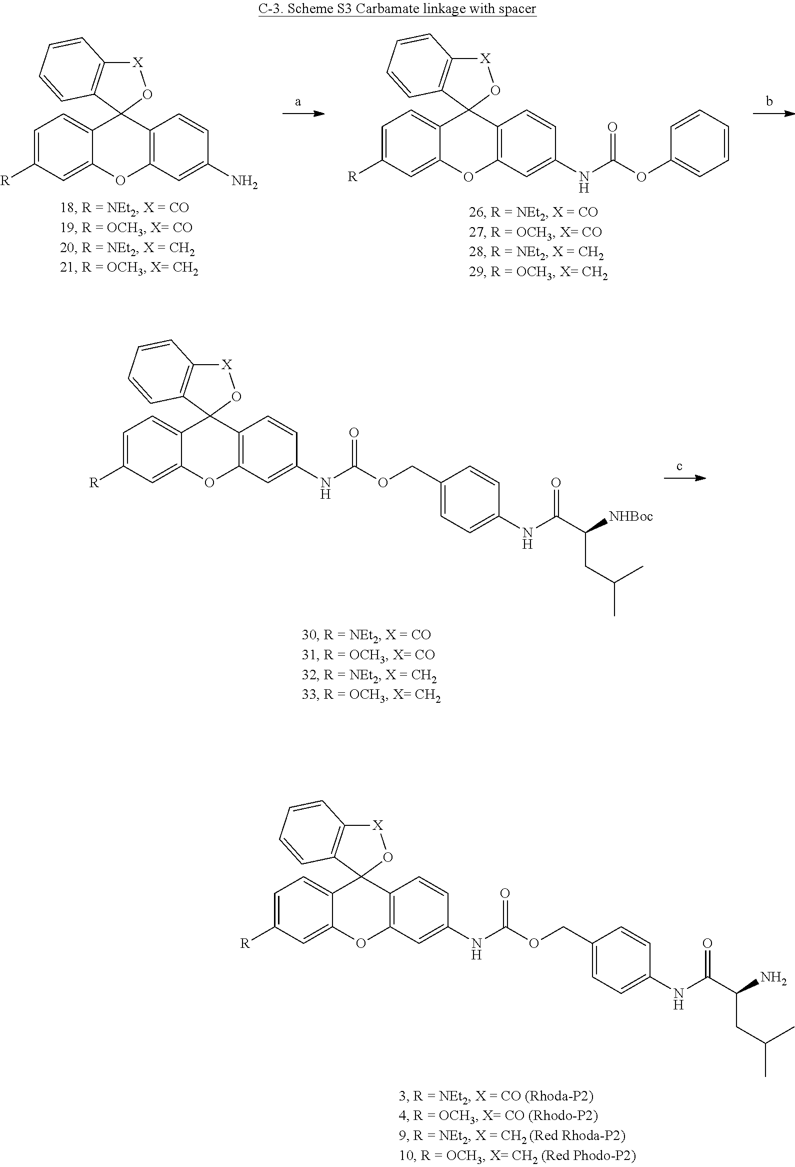

Synthesis of LAP-responsive fluorescent probes via carbamate linkage with a spacer. Reagents and conditions: (a) Phenyl chloroformate, DIPEA, CH₂Cl₂, rt, 26=57%, 27=69%, 28=64%, 29=85%; (b) 41, DBU, acetone, rt, 30=79%, 31=73%, 32=47%, 33=60%; (c) 30% TFA in CH₂Cl₂, −20° C., 3=47%, 4=78%, 9=47%, 10=48%.

### Phenyl (3′-(diethylamino)-3-oxo-3H-spiro[isobenzofuran-1,9′-xanthen]-6′-yl)carbamate (26)

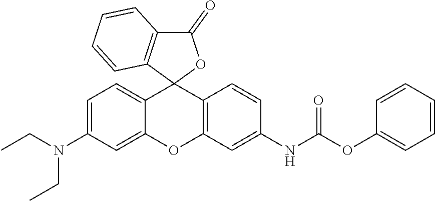 [SMILES: CCN(CC)c1ccc2c(c1)Oc1cc(NC(=O)Oc3ccccc3)ccc1C21OC(=O)c2ccccc21]

Compound 26 was synthesized by carbamate of 18 (Rhoda-NH₂, 45 mg, 0.12 mmol) with 1 M Phenyl chloroformate (0.24 mL, 0.24 mmol) and 1 M DIPEA (0.24 mL, 0.24 mmol) in CH₂Cl₂ according to the general procedure B. The crude residue was purified by flash column chromatography on silica gel (EtOAc:Hexane=1:4) to produce 26 in 57% of yield (34 mg).

¹H NMR (400 MHZ, DMSO-d₆) δ 10.53 (s, 1H), 7.99 (d, J=7.3 Hz, 1H), 7.80-7.76 (m, 1H), 7.71 (t, J=7.5 Hz, 1H), 7.54 (d, J=1.8 Hz, 1H), 7.43 (t, J=7.8 Hz, 2H), 7.28-7.26 (m, 2H), 7.24-7.22 (m, 2H), 7.19 (dd, J=8.7, 1.8 Hz, 1H), 6.70 (d, J=8.7 Hz, 1H), 6.48 (d, J=16.0 Hz, 3H), 3.37-3.32 (m, 4H), 1.08 (t, J=7.1 Hz, 6H); ¹³C NMR (100 MHz, DMSO-d₆) δ 174.9, 169.0, 153.5, 152.6, 152.4, 151.6, 149.4, 141.5, 139.1, 135.7, 131.0, 130.2, 129.2, 128.8, 128.7, 126.5, 124.8, 124.2, 119.2, 113.2, 105.2, 104.7, 97.2, 83.6, 54.1, 44.2, 44.0, 24.4, 23.4, 22.0, 12.5.

### Phenyl (3′-methoxy-3-oxo-3H-spiro[isobenzofuran-1,9′-xanthen]-6′-yl)carbamate (27)

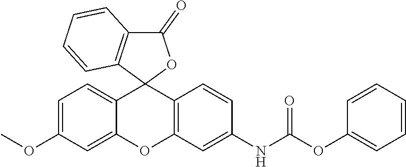 [SMILES: COc1ccc2c(c1)Oc1cc(NC(=O)Oc3ccccc3)ccc1C21OC(=O)c2ccccc21]

Compound 27 was synthesized from 19 (Rhodo-NH₂, 100 mg, 0.215 mmol) according to the general procedure B, using 1 M phenyl chloroformate (0.43 mL, 0.43 mmol) and 1 M DIPEA (0.43 mL, 0.43 mmol) in CH₂Cl₂. The residue was purified by flash column chromatography on silica gel (EtOAc:Hexane=1:2) to give 68 mg of 27 in 69% of yield.

¹H NMR (400 MHZ, DMSO-d₆) δ 10.57 (s, 1H), 8.01 (d, J=7.3 Hz, 1H), 7.80 (t, J=7.3, 1H), 7.72 (t, J=7.1 Hz, 1H), 7.60 (s, 1H), 7.47-7.39 (m, 2H), 7.28 (d, J=7.3 Hz, 2H), 7.24 (d, J=7.8 Hz, 3H), 6.99 (d, J=2.3 Hz, 1H), 6.76 (d, J=8.7 Hz, 1H), 6.71 (dd, J=8.9, 2.5 Hz, 1H), 6.68 (s, 1H), 3.80 (s, 3H); ¹³C NMR (100 MHZ, DMSO-d₆) δ 168.66, 161.11, 152.52, 151.73, 151.63, 150.99, 150.30, 140.99, 135.75, 130.24, 129.49, 129.36, 128.94, 128.74, 125.76, 125.65, 124.78, 123.96, 121.93, 118.78, 115.20, 113.03, 112.15, 110.75, 105.39, 100.86, 82.08, 55.69; HRMS (ESI⁺): m/z Calcd for C₂₈H₁₉NO₆ [M+H]⁺: 466.1246, Found: 466.1284.

### Phenyl (3′-(diethylamino)-3H-spiro[isobenzofuran-1,9′-xanthen]-6′-yl)carbamate (28)

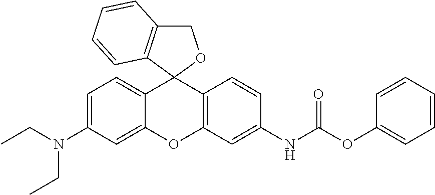 [SMILES: CCN(CC)c1ccc2c(c1)Oc1cc(NC(=O)Oc3ccccc3)ccc1C21OCc2ccccc21]

Compound 28 was synthesized from 20 (Red Rhoda-NH₂, 40 mg, 0.107 mmol) with 1 M phenyl chloroformate (0.21 mmol, 0.21 mL) and 1 M DIPEA (0.21 mmol, 0.21 mL) in CH₂Cl₂ according to the general procedure B. The crude residue was purified by flash column chromatography on silica gel (EtOAc:Hexane=1:2) to produce 28 in 64% of yield (34 mg).

¹H NMR (400 MHZ, DMSO-d₆) δ 10.39 (s, 1H), 7.44-7.41 (m, 4H), 7.35 (t, J=7.3 Hz, 1H), 7.25 (dd, J=16.7, 7.5 Hz, 4H), 7.15 (dd, J=8.7, 1.8 Hz, 1H), 6.85 (d, J=8.7 Hz, 1H), 6.77 (d, J=7.3 Hz, 1H), 6.67 (d, J=8.7 Hz, 1H), 6.42-6.39 (m, 2H), 5.21 (s, 2H), 3.31 (t, J=7.1 Hz, 3H), 1.07 (t, J=7.1 Hz, 6H); ¹³C NMR (100 MHZ, DMSO-d₆) δ 152.3, 151.6, 151.0, 150.8, 148.8, 146.0, 139.9, 139.3, 130.1, 130.0, 128.8, 128.5, 126.2, 123.8, 122.6, 121.7, 120.6, 114.3, 112.0, 108.8, 105.7, 97.4, 83.2, 72.1, 55.5, 44.3, 13.0; HRMS (ESI⁺): m/z Calcd for C₃₁H₂₈N₂O₄ [M+H]⁺: 493.2083, Found: 493.2129.

### Phenyl (3′-methoxy-3H-spiro[isobenzofuran-1,9′-xanthen]-6′-yl)carbamate (29)

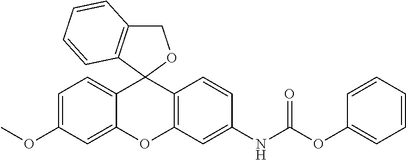 [SMILES: COc1ccc2c(c1)Oc1cc(NC(=O)Oc3ccccc3)ccc1C21OCc2ccccc21]

Compound 29 was synthesized from 21 (Red Rhodo-NH₂, 20 mg, 0.06 mmol) according to the general procedure B, using 1 M phenyl chloroformate (0.12 mmol, 0.12 mL) and 1 M DIPEA (0.12 mmol, 0.12 mL) in CH₂Cl₂. The residue was purified by flash column chromatography on silica gel (EtOAc:Hexane=1:2) to give 23 mg of compound 29 in 85% of yield.

1H-NMR (400 MHZ, DMSO-d₆) δ 10.43 (s, 1H), 7.44 (q, J=8.1 Hz, 4H), 7.36 (t, J=7.3 Hz, 1H), 7.28-7.22 (m, 4H), 7.18 (dd, J=8.7, 1.8 Hz, 1H), 6.92 (d, J=8.7 Hz, 1H), 6.87-6.84 (m, 2H), 6.76 (d, J=7.3 Hz, 1H), 6.66 (dd, J=8.7, 2.7 Hz, 1H), 5.29 (s, 2H), 3.77 (s, 3H); ¹³C-NMR (100 MHz, DMSO-d₆) δ 159.9, 151.6, 150.4, 150.4, 149.8, 145.4, 139.5, 138.3, 129.7, 129.5, 129.3, 128.3, 128.0, 125.6, 123.0, 121.9, 121.2, 119.6, 117.2, 114.2, 111.3, 105.0, 100.2, 82.4, 72.0, 55.5; HRMS (ESI⁺): m/z Calcd for C₂₈H₂₁NO₅ [M+H]⁺: 452.1453, Found: 452.1496.

### 4-((S)-2-((tert-Butoxycarbonyl)amino)-4-methylpentanamido)benzyl (3′-(diethylamino)-3-oxo-3H-spiro[isobenzofuran-1,9′-xanthen]-6′-yl)carbamate (30)

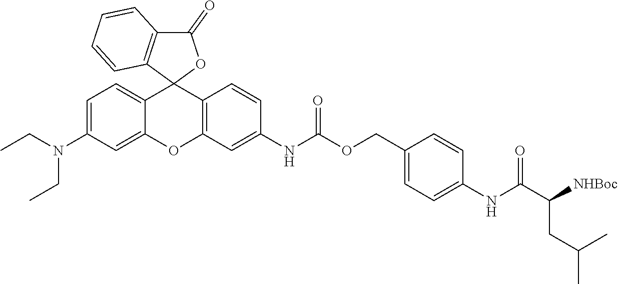 [SMILES: CCN(CC)c1ccc2c(c1)Oc1cc(NC(=O)OCc3ccc(NC(=O)[C@H](CC(C)C)NC(=O)OC(C)(C)C)cc3)ccc1C21OC(=O)c2ccccc21]

Compound 30 was synthesized from 26 (28 mg, 0.055 mmol) with 41 (19 mg, 0.055 mmol) and DBU (9 mg, 0.055 mmol) according to the general procedure B. The residue was purified by flash column chromatography on silica gel (CH₂Cl₂:MeOH=20:1) to give compound 30 in 79% of yield (31 mg).

¹H NMR (400 MHZ, DMSO-d₆) δ 10.05 (s, 1H), 10.01 (s, 1H), 7.97 (d, J=7.3 Hz, 1H), 7.77 (td, J=7.5, 1.1 Hz, 1H), 7.72-7.68 (m, 1H), 7.61 (d, J=8.2 Hz, 2H), 7.52 (d, J=1.8 Hz, 1H), 7.36 (d, J=8.2 Hz, 2H), 7.26 (d, J=7.8 Hz, 1H), 7.13 (dd, J=8.7, 2.3 Hz, 1H), 7.03 (d, J=8.2 Hz, 1H), 6.64 (d, J=8.7 Hz, 1H), 6.47 (d, J=17.8 Hz, 3H), 5.10 (s, 2H), 4.11 (dd, J=13.7, 9.1 Hz, 1H), 3.35 (t, J=7.1 Hz, 4H), 1.62 (td, J=13.4, 6.7 Hz, 1H), 1.55-1.48 (m, 1H), 1.41 (dd, J=8.5, 5.3 Hz, 1H), 1.36 (s, 9H), 1.08 (t, J=7.1 Hz, 6H), 0.88 (q, J=3.2 Hz, 6H); ¹³C NMR (100 MHZ, DMSO-d₆) δ 171.9, 168.8, 158.2, 155.5, 153.3, 152.4, 152.2, 151.4, 149.2, 141.2, 139.0, 135.5, 130.9, 130.0, 129.0, 128.6, 128.5, 126.3, 124.5, 124.0, 119.1, 114.0, 112.9, 108.6, 105.0, 104.5, 97.0, 83.4, 78.0, 65.8, 53.5, 43.7, 40.6, 24.3, 23.0, 21.5, 12.3; HRMS (ESI⁺): m/z Calcd for C₄₃H₄₈N₄O₈ [M+H]⁺: 749.3506, Found: 749.3558.

### 4-((S)-2-((tert-Butoxycarbonyl)amino)-4-methylpentanamido)benzyl (3′-methoxy-3-oxo-3H-spiro[isobenzofuran-1,9′-xanthen]-6′-yl)carbamate (31)

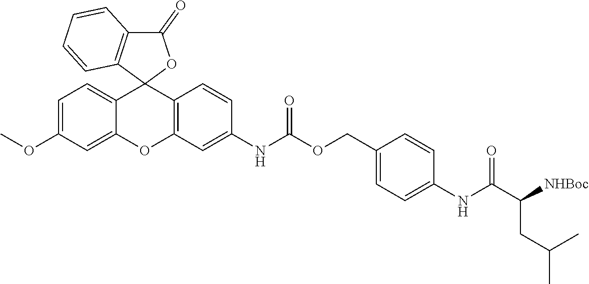 [SMILES: COc1ccc2c(c1)Oc1cc(NC(=O)OCc3ccc(NC(=O)[C@H](CC(C)C)NC(=O)OC(C)(C)C)cc3)ccc1C21OC(=O)c2ccccc21]

Compound 31 was synthesized by the reaction of intermediate 27 (50 mg, 0.11 mmol) with benzyl alcohol 41 (37 mg, 0.11 mmol) and DBU (17 mg, 0.11 mmol) according to the general procedure B. The crude residue was purified by flash column chromatography on silica gel (EtOAc:Hexane=2:3) to produce 31 in 73% of yield (57 mg).

¹H NMR (400 MHZ, DMSO-d₆) δ 10.09 (s, 1H), 10.01 (s, 1H), 8.00 (d, J=7.8 Hz, 1H), 7.78 (t, J=7.5 Hz, 1H), 7.71 (t, J=7.3 Hz, 1H), 7.62 (d, J=8.2 Hz, 2H), 7.57 (d, J=1.4 Hz, 1H), 7.36 (d, J=8.2 Hz, 2H), 7.26 (d, J=7.8 Hz, 1H), 7.16 (dd, J=8.7, 1.8 Hz, 1H), 7.03 (d, J=7.8 Hz, 1H), 6.97 (d, J=2.3 Hz, 1H), 6.70 (q, J=4.1 Hz, 2H), 6.65 (d, J=8.7 Hz, 1H), 5.10 (s, 2H), 4.11 (m, 1H), 3.80 (s, 3H), 1.62 (q, J=6.4 Hz, 1H), 1.57-1.45 (m, 1H), 1.45-1.25 (m, 10H), 0.88 (q, J=3.2 Hz, 6H); ¹³C NMR (100 MHz, DMSO-d₆) δ 171.89, 168.66, 161.09, 155.46, 153.29, 152.50, 151.78, 150.98, 141.47, 139.04, 135.73, 130.87, 130.22, 129.03, 128.57, 125.80, 124.76, 123.97, 119.09, 114.45, 112.44, 112.07, 110.79, 105.00, 100.85, 82.18, 78.00, 65.86, 55.68, 53.50, 40.60, 28.19, 24.34, 22.97, 21.53; HRMS (ESI⁺): m/z Calcd for C₄₀H₄₁N₃O₉ [M+H]⁺: 708.2876, Found: 708.2926.

### 4-((S)-2-((tert-Butoxycarbonyl)amino)-4-methylpentanamido)benzyl (3′-(diethylamino)-3H-spiro[isobenzofuran-1,9′-xanthen]-6′-yl)carbamate (32)

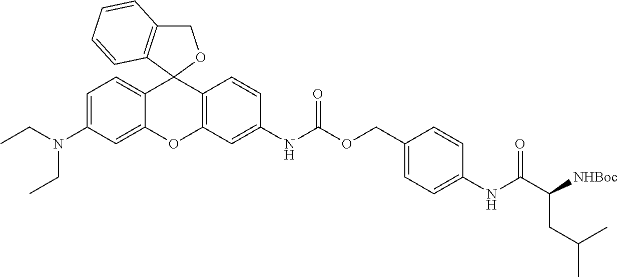 [SMILES: CCN(CC)c1ccc2c(c1)Oc1cc(NC(=O)OCc3ccc(NC(=O)[C@H](CC(C)C)NC(=O)OC(C)(C)C)cc3)ccc1C21OCc2ccccc21]

Compound 32 was synthesized from intermediate 28 (30 mg, 0.061 mmol) with benzyl alcohol 41 (21 mg, 0.061 mmol) and DBU (10 mg, 0.061 mmol) according to the general procedure B. The residue was purified by flash column chromatography on silica gel (CH₂Cl₂:MeOH=30:1) to give 32 in 47% of yield (21 mg).

¹H NMR (400 MHZ, DMSO-d₆) δ 8.08 (s, 1H), 7.71 (d, J=7.3 Hz, 2H), 7.56-7.48 (m, 1H), 7.40 (d, J=7.8 Hz, 2H), 7.37-7.30 (m, 1H), 7.09 (dd, J=9.6, 2.7 Hz, 1H), 6.81-6.72 (m, 2H), 6.58 (d, J=8.7 Hz, 1H), 6.48 (d, J=8.7 Hz, 1H), 6.30 (q, J=2.4 Hz, 2H), 5.29 (s, 2H), 5.11 (s, 2H), 3.36 (d, J=14.6 Hz, 4H), 3.16 (d, J=5.5 Hz, 1H), 1.60-1.39 (m, 1H), 1.39-1.27 (m, 1H), 1.25 (m, 1H), 1.22 (s, 9H), 1.07 (t, J=6.9 Hz, 6H), 0.91-0.78 (m, 6H); ¹³C NMR (100 MHZ, DMSO-d₆) δ 172.9, 168.8, 155.5, 153.3, 152.4, 152.2, 151.4, 147.2, 140.5, 139.2, 132.5, 130.9, 130.0, 129.0, 128.6, 128.5, 125.3, 124.5, 122.3, 119.8, 116.3, 112.4, 108.6, 105.1, 104.8, 97.1, 83.4, 81.1, 66.0, 54.0, 44.2, 42.6, 28.5, 24.5, 23.4, 21.9, 12.5; HRMS (ESI⁺): m/z Calcd for C₄₃H₅₀N₄O₇ [M+H]⁺: 735.3713, Found: 735.3771.

### 4-((S)-2-((tert-Butoxycarbonyl)amino)-4-methylpentanamido)benzyl(3′-methoxy-3H-spiro[isobenzofuran-1,9′-xanthen]-6′-yl)carbamate (33)

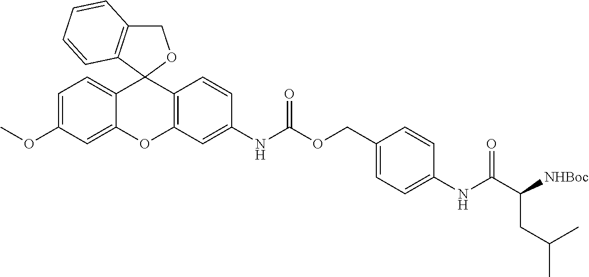 [SMILES: COc1ccc2c(c1)Oc1cc(NC(=O)OCc3ccc(NC(=O)[C@H](CC(C)C)NC(=O)OC(C)(C)C)cc3)ccc1C21OCc2ccccc21]

Compound 33 was synthesized by the reaction of intermediate 29 (35 mg, 0.078 mmol) with benzyl alcohol 41 (26 mg, 0.078 mmol) and DBU (12 mg, 0.078 mmol) according to the general procedure B. The crude residue was purified by flash column chromatography on silica gel (CH₂Cl₂:EA=20:1) to produce compound 33 in 60% of yield (32 mg).

¹H NMR (400 MHZ, DMSO-d₆) δ 10.03 (s, 1H), 9.94 (s, 1H), 7.62 (d, J=8.7 Hz, 2H), 7.45 (d, J=6.9 Hz, 2H), 7.35 (t, J=7.1 Hz, 3H), 7.22 (t, J=7.3 Hz, 1H), 7.12 (dd, J=8.7, 1.8 Hz, 1H), 7.04 (d, J=7.8 Hz, 1H), 6.76-6.73 (m, 3H), 6.60 (dd, J=8.7, 2.3 Hz, 1H), 6.54 (d, J=8.2 Hz, 1H), 5.27 (s, 2H), 5.09 (s, 2H), 4.14-4.08 (m, 1H), 3.76 (s, 3H), 1.62 (q, J=6.6 Hz, 1H), 1.55-1.48 (m, 1H), 1.42 (dd, J=8.5, 5.3 Hz, 1H), 1.36 (s, 9H), 0.88 (q, J=3.1 Hz, 6H); ¹³C NMR (100 MHZ, DMSO-d₆) δ 173.3, 159.9, 155.5, 153.3, 150.7, 149.8, 145.5, 140.0, 139.0, 138.3, 129.7, 129.3, 129.0, 128.3, 128.1, 123.0, 121.0, 119.1, 117.6, 110.7, 100.1, 82.8, 78.0, 71.2, 65.7, 56.8, 55.4, 53.5, 44.4, 40.6, 28.2, 24.3, 23.0, 21.5; HRMS (ESI⁺): m/z Calcd for C₄₀H₄₃N₃O₈ [M+H]⁺: 694.3084, Found: 694.3133.

### 4-((S)-2-Amino-4-methylpentanamido)benzyl (3′-(diethylamino)-3-oxo-3H-spiro[isobenzofuran-1,9′-xanthen]-6′-yl)carbamate (3, Rhoda-P2)

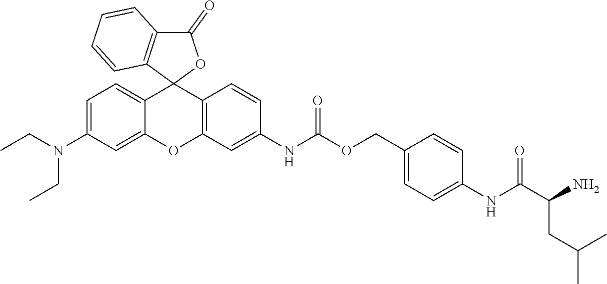 [SMILES: CCN(CC)c1ccc2c(c1)Oc1cc(NC(=O)OCc3ccc(NC(=O)[C@@H](N)CC(C)C)cc3)ccc1C21OC(=O)c2ccccc21]

Probe 3 (Rhoda-P2) was synthesized from 30 (30 mg, 0.043 mmol) according to the general procedure D, using 30% trifluoroacetic acid in CH2Cl2 (1.8 mL). The crude residue was purified by flash column chromatography on silica gel (CH₂Cl₂:MeOH=5:1) to give 12 mg of probe 3 Rhoda-P2) in 47% of yield.

¹H NMR (400 MHZ, DMSO-d₆) δ 10.01 (s, 1H), 7.94 (d, J=7.3 Hz, 1H), 7.73 (t, J=7.3 Hz, 1H), 7.67 (d, J=7.3 Hz, 1H), 7.61 (d, J=8.7 Hz, 2H), 7.48 (s, 1H), 7.32 (d, J=8.7 Hz, 2H), 7.21 (d, J=7.8 Hz, 1H), 7.09 (dd, J=8.7, 1.8 Hz, 1H), 6.60 (d, J=8.7 Hz, 1H), 6.43 (d, J=16.9 Hz, 3H), 5.06 (s, 2H), 3.31 (m, 4H), 2.45 (t, J=1.6 Hz, 1H), 1.73-1.66 (m, 1H), 1.46-1.39 (m, 1H), 1.31-1.24 (m, 1H), 1.04 (t, J=7.1 Hz, 6H), 0.84 (dd, J=10.1, 6.4 Hz, 6H); ¹³C NMR (100 MHz, DMSO-d₆) δ 172.54, 168.68, 161.10, 155.51, 152.57, 151.79, 150.82, 141.16, 135.76, 130.24, 128.95, 128.48, 125.76, 124.76, 123.97, 115.40, 113.15, 112.13, 110.72, 106.24, 100.85, 82.13, 78.08, 55.69, 53.65, 28.19, 24.34, 22.93, 21.52; HRMS (ESI⁺): m/z Calcd for C₃₈H₄₀N₄O₆ [M+H]⁺: 649.2981, Found: 649.3028.

### 4-((S)-2-Amino-4-methylpentanamido)benzyl methoxy-3-oxo-3H-spiro[isobenzofuran-1,9′-xanthen]-6′-(3′-yl)carbamate (4, Rhodo-P2)

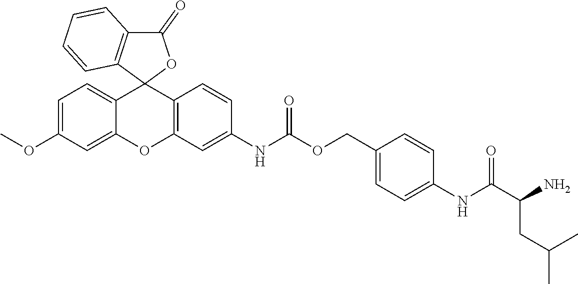 [SMILES: COc1ccc2c(c1)Oc1cc(NC(=O)OCc3ccc(NC(=O)[C@@H](N)CC(C)C)cc3)ccc1C21OC(=O)c2ccccc21]

Probe 4 (Rhodo-P2) was synthesized of compound 31 (20 mg, 0.028 mmol) according to the general procedure D. The crude residue was purified by flash column chromatography on silica gel (CH₂Cl₂:EA=10:1) to give probe 4 (Rhodo-P2) in 78% of yield (22 mg).

1H-NMR (400 MHZ, DMSO-d₆) δ 10.08 (s, 1H), 8.00 (d, J=7.3 Hz, 1H), 7.78 (t, J=7.1 Hz, 1H), 7.71 (t, J=7.3 Hz, 1H), 7.65 (d, J=8.7 Hz, 2H), 7.58 (s, 1H), 7.36 (d, J=8.7 Hz, 2H), 7.26 (d, J=7.8 Hz, 1H), 7.16 (d, J=8.7 Hz, 1H), 6.97 (d, J=2.3 Hz, 1H), 6.75-6.62 (m, 3H), 5.10 (s, 2H), 3.80 (s, 3H), 1.80-1.67 (m, 1H), 1.51-1.41 (m, 1H), 1.36-1.26 (m, 1H), 0.88 (dd, J=10.1, 6.4 Hz, 6H); ¹³CNMR (100 MHZ, DMSO-d₆) δ 174.90, 168.65, 161.08, 153.29, 152.49, 151.77, 150.97, 138.94, 135.71, 130.78, 130.21, 129.02, 128.93, 128.56, 125.80, 124.75, 123.96, 119.01, 114.43, 112.44. 112.05, 110.79, 104.98, 100.85, 82.17, 65.88, 55.68, 53.93, 44.07, 24.21, 23.20, 21.82; HRMS (ESI⁺): m/z Calcd for C₃₅H₃₃N₃O₇ [M+H]⁺: 608.2352, Found: 608.2404.

### 4-((S)-2-Amino-4-methylpentanamido)benzyl (3′-(diethylamino)-3H-spiro[isobenzofuran-1,9′-xanthen]-6′-yl)carbamate (9, Red Rhoda-P2)

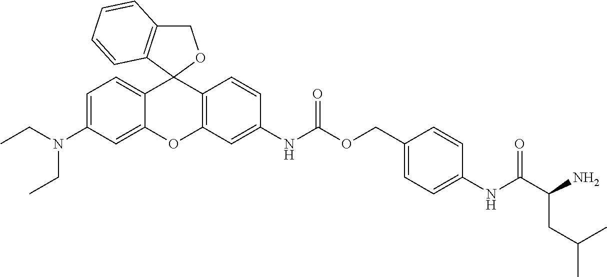 [SMILES: CCN(CC)c1ccc2c(c1)Oc1cc(NC(=O)OCc3ccc(NC(=O)[C@@H](N)CC(C)C)cc3)ccc1C21OCc2ccccc21]

Probe 9 (Red Rhoda-P2) was synthesized from compound 32 (15 mg, 0.02 mmol) according to the general procedure D, using 30% trifluoroacetic acid in CH₂Cl₂ (0.9 mL). The residue was purified by flash column chromatography on silica gel (CH₂Cl₂:MeOH=5:1) to give 6 mg of probe 9 (Red Rhoda-P2) in 47% of yield.

¹H NMR (400 MHZ, DMSO-d₆) δ 9.88 (s, 1H), 7.64 (dd, J=6.9, 1.8 Hz, 2H), 7.43 (d, J=7.3 Hz, 1H), 7.38-7.32 (m, 4H), 7.23 (t, J=7.1 Hz, 1H), 7.09 (dd, J=8.5, 2.1 Hz, 1H), 6.77 (dd, J=12.8, 8.2 Hz, 2H), 6.65 (d, J=8.7 Hz, 1H), 6.42-6.37 (m, 2H), 5.21 (d, J=13.3 Hz, 2H), 5.10 (d, J=12.3 Hz, 2H), 3.34 (d, J=6.9 Hz, 4H), 3.16 (s, 1H), 1.80-1.69 (m, 1H), 1.49-1.42 (m, 1H), 1.34-1.27 (m, 1H), 1.07 (t, J=6.9 Hz, 6H), 0.88 (dd, J=10.5, 6.4 Hz, 6H); ¹³C NMR (100 MHZ, DMSO-d₆) δ 175.5, 151.5, 150.7, 148.7, 145.9, 139.5, 139.3, 130.0, 129.7, 129.6, 128.7, 128.3, 123.7, 121.6, 119.5, 119.9, 111.9, 108.6, 105.2, 97.3, 83.2, 66.2, 54.5, 44.6, 44.2, 24.7, 23.8, 22.4, 12.9; HRMS (ESI⁺): m/z Calcd for C₃₈H₄₂N₄O₅[M+H]⁺: 635.3189, Found: 635.3226.

### 4-((S)-2-Amino-4-methylpentanamido)benzyl (3′-methoxy-3H-spiro[isobenzofuran-1,9′-xanthen]-6′-yl)carbamate (10, Red Rhodo-P2)

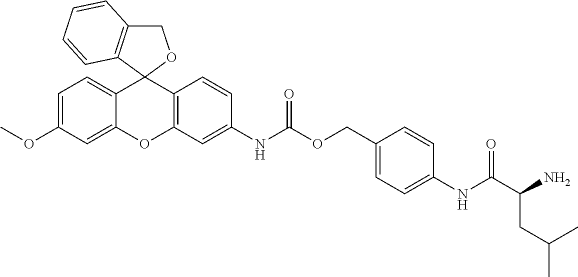 [SMILES: COc1ccc2c(c1)Oc1cc(NC(=O)OCc3ccc(NC(=O)[C@@H](N)CC(C)C)cc3)ccc1C21OCc2ccccc21]

Probe 10 (Red Rhodo-P2) was synthesized of compound 33 (25 mg, 0.042 mmol) according to the general procedure D. The residue was purified by flash column chromatography on silica gel (CH₂Cl₂:EA=10:1) to give probe 10 (Red Rhodo-P2) in 48% of yield (10 mg).

¹H NMR (400 MHZ, DMSO-d₆) δ 9.94 (s, 1H), 7.65 (d, J=8.7 Hz, 2H), 7.45 (d, J=7.3 Hz, 2H), 7.37-7.33 (m, 3H), 7.22 (t, J=7.1 Hz, 1H), 7.12 (dd, J=8.5, 2.1 Hz, 1H), 6.87-6.82 (m, 3H), 6.74 (d, J=7.8 Hz, 1H), 6.66 (dd, J=8.7, 2.7 Hz, 1H), 5.27 (s, 2H), 5.09 (s, 2H), 4.10 (d, J=7.3 Hz, 1H), 3.77 (s, 3H), 1.79-1.69 (m, 1H), 1.48-1.42 (m, 1H), 1.34-1.27 (m, 1H), 0.88 (dd, J=10.5, 6.4 Hz, 6H); 13c NMR (100 MHZ, DMSO-d₆) δ 175.0, 159.9, 153.3, 150.5, 149.7, 145.4, 140.0, 138.9, 138.3, 130.9, 129.7, 129.2, 129.0, 128.3, 128.0, 123.0, 121.2, 119.0, 117.2, 111.2, 100.2, 82.4, 71.9, 65.8, 55.5, 54.9, 54.0, 48.6, 44.1, 24.2, 23.2, 21.8; HRMS (ESI⁺): m/z Calcd for C₃₅H₃₅N₃O₆ [M+H]⁺: 594.2559, Found: 594.2597.

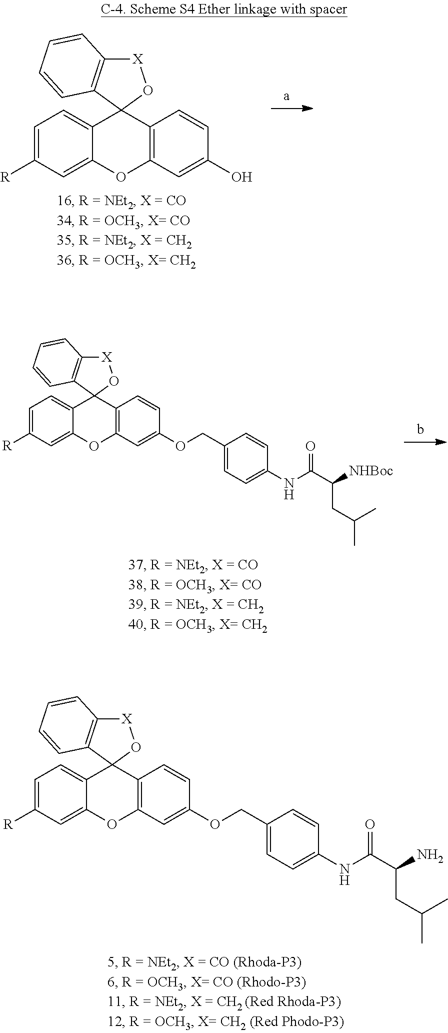

Synthesis of LAP-responsive fluorescent probes via ether linkage with a spacer. Reagents and conditions: (a) 42, Ag2O, toluene, 120° C., 37=52%, 38=49%, 39=24%, 40=22%; (b) 30% TFA in CH2Cl2, −20° C., 5=58%, 6=42%, 11=59%, 12=48%.

### tert-Butyl ((2S)-1-((4-(((3′-(diethylamino)-3-oxo-3H-spiro[isobenzofuran-1,9′-xanthen]-6′-yl)oxy)methyl)phenyl)amino)-4-methyl-1-oxopentan-2-yl)carbamate (37)

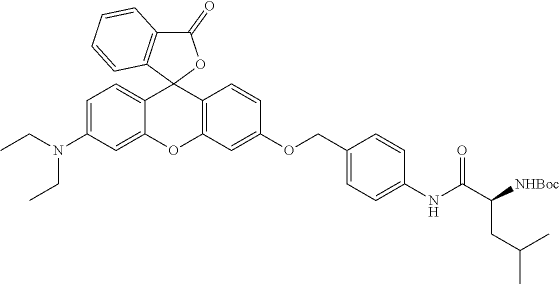 [SMILES: CCN(CC)c1ccc2c(c1)Oc1cc(OCc3ccc(NC(=O)[C@H](CC(C)C)NC(=O)OC(C)(C)C)cc3)ccc1C21OC(=O)c2ccccc21]

Compound 37 was synthesized by the alkylation of 16 (Rhodo-OH, 50 mg, 0.129 mmol) with benzyl bromide 42 (62 mg, 0.155 mmol) and Ag²O (45 mg, 0.194 mmol) according to the general procedure C. The crude residue was purified by flash column chromatography on silica gel (CH₂Cl₂:MeOH=20:1) to produce 37 in 52% of yield (47 mg).

¹H NMR (400 MHZ, DMSO-d₆) δ 10.00 (s, 1H), 7.98 (d, J=7.3 Hz, 1H), 7.77 (t, J=7.5 Hz, 1H), 7.70 (t, J=7.5 Hz, 1H), 7.62 (d, J=8.7 Hz, 2H), 7.38 (d, J=8.2 Hz, 2H), 7.26 (d, J=7.8 Hz, 1H), 7.03 (d, J=7.8 Hz, 1H), 6.93 (d, J=2.3 Hz, 1H), 6.73 (dd, J=8.7, 2.3 Hz, 1H), 6.61 (d, J=8.7 Hz, 1H), 6.45 (d, J=1.8 Hz, 3H), 5.09 (s, 2H), 4.11 (dd, J=14.4, 8.0 Hz, 1H), 3.40-3.35 (m, 4H), 1.64-1.58 (m, 1H), 1.55-1.48 (m, 1H), 1.42 (dd, J=8.7, 5.5 Hz, 1H), 1.36 (s, 9H), 1.08 (t, J=7.1 Hz, 6H), 0.88 (q, J=3.4 Hz, 6H); ¹³C NMR (100 MHZ, DMSO-d₆) δ 168.7, 159.9, 155.4, 152.4, 152.2, 152.1, 149.2, 138.9, 135.5, 131.1, 130.0, 128.9, 128.6, 128.4, 126.3, 124.5, 124.0, 119.2, 112.2, 111.5, 104.5, 101.6, 96.9, 83.5, 78.0, 69.4, 53.5, 43.8, 40.6, 28.2, 24.3, 22.9, 21.6, 12.3; HRMS (ESI⁺): m/z Calcd for C₄₂H₄₇N₃O₇ [M+H]⁺: 706.3448, Found: 706.3492.

### tert-Butyl ((2S)-1-((4-(((3′-methoxy-3-oxo-3H-spiro[isobenzofuran-1,9′-xanthen]-6′-yl)oxy)methyl)phenyl)amino)-4-methyl-1-oxopentan-2-yl)carbamate (38)

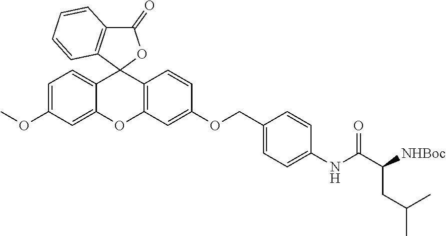 [SMILES: COc1ccc2c(c1)Oc1cc(OCc3ccc(NC(=O)[C@H](CC(C)C)NC(=O)OC(C)(C)C)cc3)ccc1C21OC(=O)c2ccccc21]

Compound 38 was synthesized by the reaction of 34 (Fluor-OH, 50 mg, 0.144 mmol) with benzyl bromide 42 (69 mg, 0.173 mmol) and Ag₂O (51 mg, 0.216 mmol) according to the general procedure C. The crude residue was purified by flash column chromatography on silica gel (EtOAc:Hexane=1:2) to produce 38 in 49% of yield (47 mg).

¹H NMR (400 MHZ, DMSO-d₆) δ 9.98 (s, 1H), 8.00 (d, J=7.3 Hz, 1H), 7.78 (t, J=7.3 Hz, 1H), 7.71 (t, J=7.5 Hz, 1H), 7.62 (d, J=8.2 Hz, 2H), 7.38 (d, J=8.7 Hz, 2H), 7.26 (d, J=7.3 Hz, 1H), 7.05-6.95 (m, 2H), 6.92 (d, J=2.3 Hz, 1H), 6.78 (dd, J=8.7, 2.3 Hz, 1H), 6.71 (dd, J=8.9, 2.5 Hz, 1H), 6.66 (dd, J=8.9, 2.1 Hz, 2H), 5.11 (s, 2H), 4.10 (t, J=7.1 Hz, 1H), 3.81 (s, 3H), 2.07 (s, 9H), 1.63 (t, J=6.2 Hz, 1H), 1.59-1.46 (m, 1H), 0.88 (q, J=3.2 Hz, 9H); ¹³C NMR (100 MHZ, DMSO-d₆) δ 206.44, 171.80, 168.57, 161.05, 160.08, 152.43, 151.74, 151.66, 138.82, 135.67, 131.10, 130.19, 128.95, 128.40, 125.86, 124.69, 123.94, 119.22, 112.67, 111.98, 111.03, 110.83, 101.65, 100.77, 82.30, 77.99, 69.47, 55.63, 53.52, 37.91, 30.64, 28.16, 24.32, 22.90, 21.54; HRMS (ESI⁺): m/z Calcd for C₃₉H₄₀N₂O₈ [M+H]⁺: 665.2818, Found: 665.2861.

### tert-Butyl ((2S)-1-((4-(((3′-(diethylamino)-3H-spiro[isobenzofuran-1,9′-xanthen]-6′-yl)oxy)methyl)phenyl)amino)-4-methyl-1-oxopentan-2-yl)carbamate (39)

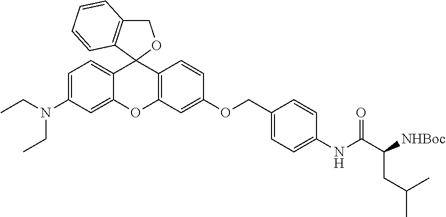 [SMILES: CCN(CC)c1ccc2c(c1)Oc1cc(OCc3ccc(NC(=O)[C@H](CC(C)C)NC(=O)OC(C)(C)C)cc3)ccc1C21OCc2ccccc21]

Compound 39 was synthesized by the alkylation of 35 (Red Rhodo-OH, 40 mg, 0.107 mmol) with benzyl bromide 42 (51 mg, 0.128 mmol) and Ag₂O (37 mg, 0.161 mmol) according to the general procedure C. The crude residue was purified by flash column chromatography on silica gel (CH₂Cl₂:MeOH=30:1) to produce 39 in 24% of yield (18 mg).

¹H NMR (400 MHZ, DMSO-d₆) δ 9.99 (s, 1H), 7.61 (d, J=8.7 Hz, 2H), 7.43 (d, J=7.8 Hz, 1H), 7.37 (d, J=8.2 Hz, 2H), 7.33 (d, J=7.3 Hz, 1H), 7.23 (t, J=7.3 Hz, 1H), 7.02 (d, J=7.8 Hz, 1H), 6.77 (td, J=8.1, 2.9 Hz, 3H), 6.69-6.63 (m, 2H), 6.40 (dd, J=8.7, 2.3 Hz, 1H), 6.34 (d, J=2.3 Hz, 1H), 5.18 (s, 2H), 5.05 (s, 2H), 4.11 (q, J=7.2 Hz, 1H), 3.42-3.37 (m, 4H), 3.29 (m, 1H), 1.66-1.59 (m, 1H), 1.55-1.48 (m, 1H), 1.44-1.40 (m, 1H), 1.36 (s, 9H), 1.07 (t, J=7.1 Hz, 6H), 0.88 (q, J=3.4 Hz, 6H); ¹³C NMR (100 MHZ, DMSO-d₆) δ 171.9, 158.7, 155.5, 151.0, 148.1, 145.3, 138.8, 131.4, 129.8, 129.6, 128.4, 128.2, 127.8, 119.2, 117.7, 111.4, 96.7, 82.7, 78.0, 69.2, 67.2, 63.1, 53.5, 49.5, 43.7, 40.6, 29.0, 28.2, 24.4, 23.0, 21.6, 12.4; HRMS (ESI⁺): m/z Calcd for C₄₂H₄₉N₃O₆ [M+H]⁺: 692.3655, Found: 692.3702.

### tert-Butyl ((2S)-1-((4-(((3′-methoxy-3H-spiro[isobenzofuran-1,9′-xanthen]-6′-yl)oxy)methyl)phenyl)amino)-4-methyl-1-oxopentan-2-yl)carbamate (40)

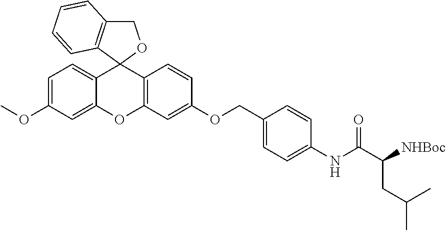 [SMILES: COc1ccc2c(c1)Oc1cc(OCc3ccc(NC(=O)[C@H](CC(C)C)NC(=O)OC(C)(C)C)cc3)ccc1C21OCc2ccccc21]

Compound 40 was synthesized by the reaction of 36 (Red Fluor-OH, 100 mg, 0.30 mmol) with benzyl bromide 42 (144 mg, 0.36 mmol) and Ag₂O (104 mg, 0.45 mmol) according to the general procedure C. The crude residue was purified by flash column chromatography on silica gel (CH₂Cl₂:MeOH=20:1) to produce 40 in 22% of yield (43 mg).

¹H NMR (400 MHZ, DMSO-d₆) δ 10.00 (s, 1H), 7.61 (d, J=8.7 Hz, 1H), 7.61 (D, J=8.7 Hz, 2H), 7.36 (q, J=7.3 Hz, 3H), 7.23 (t, J=7.3 Hz, 1H), 7.03 (d, J=8.2 Hz, 1H), 6.83 (q, J=4.3 Hz, 3H), 6.65 (dd, J=8.7, 2.7 Hz, 1H), 6.56 (d, J=2 Hz, 1H), 6.50 (dd, J=8.5, 2.5 Hz, 1H), 5.25 (s, 2H), 5.07 (s, 2H), 4.10 (d, J=15.1 Hz, 1H), 3.77 (s, 3H), 1.60 (d, J=7.3 Hz, 1H), 1.58-1.46 (m, 1H), 1.41 (d, J=8.2 Hz, 1H), 1.37 (s, 9H), 0.88 (q, J=3.2 Hz, 6H); ¹³C NMR (100 MHZ, DMSO-d₆) δ 159.56, 157.90, 150.34, 150.26, 145.13, 138.28, 129.58, 129.50, 128.00, 127.70, 122.88, 120.90, 117.09, 115.59, 111.74, 110.81, 101.36, 99.90, 82.33, 71.37, 56.21, 40.39, 27.98, 24.82, 24.14, 21.34; HRMS (ESI⁺): m/z Calcd for C₃₉H₄₂N₂O₇ [M+H]⁺: 651.3026, Found: 651.3067.

### (2S)-2-Amino-N-(4-(((3′-(diethylamino)-3-oxo-3H-spiro[isobenzofuran-1,9′-xanthen]-6′-yl)oxy)methyl)phenyl)-4-methylpentanamide (5, Rhodo-P3)

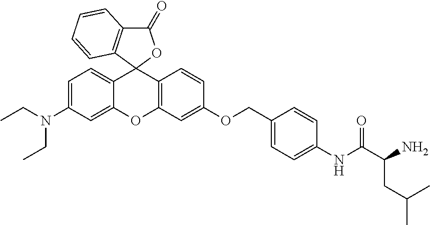 [SMILES: CCN(CC)c1ccc2c(c1)Oc1cc(OCc3ccc(NC(=O)[C@@H](N)CC(C)C)cc3)ccc1C21OC(=O)c2ccccc21]

Probe 5 (Rhodo-P3) was synthesized from 37 (40 mg, 0.057 mmol) according to the general procedure D, using 30% trifluoroacetic acid in CH₂Cl₂ (2.4 mL). The crude residue was purified by flash column chromatography on silica gel (CH₂Cl₂:MeOH=5:1) to give 20 mg of probe 5 (Rhodo-P3) in 58% of yield.

¹H NMR (400 MHZ, DMSO-d₆) δ 7.98 (d, J=7.8 Hz, 1H), 7.77 (td, J=7.4, 1.1 Hz, 1H), 7.70 (td, J=7.5, 0.9 Hz, 1H), 7.65 (d, J=8.2 Hz, 2H), 7.38 (d, J=8.7 Hz, 2H), 7.26 (d, J=7.8 Hz, 1H), 6.93 (d, J=2.3 Hz, 1H), 6.73 (dd, J=8.9, 2.5 Hz, 1H), 6.61 (d, J=8.7 Hz, 1H), 6.45 (d, J=2.7 Hz, 3H), 5.09 (s, 2H), 3.36-3.29 (m, 5H), 1.79-1.69 (m, 1H), 1.49-1.42 (m, 1H), 1.37-1.27 (m, 1H), 1.08 (t, J=7.1 Hz, 6H), 0.88 (dd, J=10.5, 6.4 Hz, 6H); ¹³C NMR (100 MHZ, DMSO-d₆) δ 174.9, 168.7, 159.9, 152.4, 152.2, 152.1, 149.2, 138.8, 135.5, 131.1, 130.0, 128.9, 128.6, 128.4, 126.4, 124.5, 124.0, 119.1, 112.2, 111.5, 108.6, 104.5, 101.6, 96.9, 83.5, 69.4, 54.0, 44.1, 43.8, 24.2, 23.2, 21.9, 12.3; HRMS (ESI⁺): m/z Calcd for C₃₇H₃₉N₃O₅ [M+H]⁺: 606.2923, Found: 606.2973.

### (2S)-2-Amino-N-(4-(((3′-methoxy-3-oxo-3H-spiro[isobenzofuran-1,9′-xanthen]-6′-yl)oxy)methyl)phenyl)-4-methylpentanamide (6, Fluor-P3)

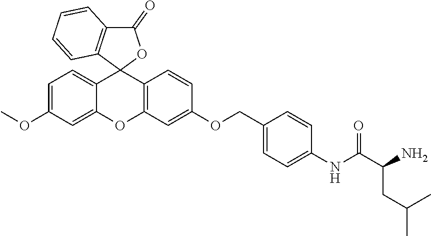 [SMILES: COc1ccc2c(c1)Oc1cc(OCc3ccc(NC(=O)[C@@H](N)CC(C)C)cc3)ccc1C21OC(=O)c2ccccc21]

Probe 6 (Fluor-P3) was synthesized from 38 (20 mg, 0.03 mmol) according to the general procedure D, using 30% trifluoroacetic acid in CH₂Cl₂ (1.2 mL). The crude residue was purified by flash column chromatography on silica gel (CH₂Cl₂:MeOH=15:1) to give 7 mg of probe 6 (Fluor-P3) in 42% of yield.

¹H NMR (400 MHZ, DMSO-d₆) δ 7.97 (d, J=7.3 Hz, 1H), 7.74 (t, J=7.1 Hz, 1H), 7.68 (t, J=7.3 Hz, 1H), 7.61 (d, J=8.2 Hz, 2H), 7.35 (d, J=8.7 Hz, 2H), 7.23 (d, J=7.3 Hz, 1H), 6.91 (dd, J=19.4, 2.5 Hz, 2H), 6.74 (dd, J=8.7, 2.7 Hz, 1H), 6.69-6.62 (m, 3H), 5.07 (s, 2H), 3.77 (s, 3H), 3.34-3.31 (m, 1H), 1.68 (td, J=13.7, 6.5 Hz, 1H), 1.48-1.40 (m, 1H), 1.33-1.26 (m, 1H), 0.84 (dd, J=9.8, 6.6 Hz, 6H); ¹³C NMR (100 MHZ, DMSO-d₆) δ 168.6, 161.1, 160.1, 152.5, 151.7, 151.7, 138.7, 135.7, 131.1, 130.2, 129.0, 128.5, 125.9, 124.7, 124.0, 119.1, 112.7, 112.0, 111.0, 110.8, 101.6, 100.8, 82.3, 69.5, 55.7, 53.8, 43.8, 42.1, 40.4, 29.0, 24.2, 23.1, 21.9; HRMS (ESI⁺): m/z Calcd for C₃₄H₃₂N₂O₆ [M+H]⁺: 565.2294, Found: 565.2334.

### (2S)-2-Amino-N-(4-(((3′-(diethylamino)-3H-spiro[isobenzofuran-1,9′-xanthen]-6′-yl)oxy)methyl)phenyl)-4-methylpentanamide (11, Red Rhodo-P3)

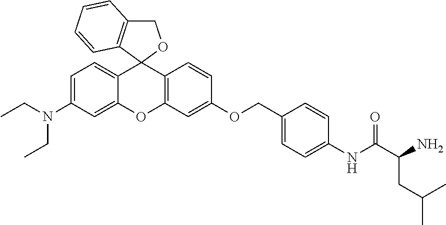 [SMILES: CCN(CC)c1ccc2c(c1)Oc1cc(OCc3ccc(NC(=O)[C@@H](N)CC(C)C)cc3)ccc1C21OCc2ccccc21]

Probe 11 (Red Rhodo-P3) was synthesized from 39 (20 mg, 0.029 mmol) according to the general procedure D, using 30% trifluoroacetic acid in CH₂Cl₂ (1.2 mL). The crude residue was purified by flash column chromatography on silica gel (CH₂Cl₂:MeOH=5:1) to give 10 mg of probe 11 (Red Rhodo-P3) in 59% of yield.

¹H NMR (400 MHZ, DMSO-d₆) δ 7.65 (d, J=8.2 Hz, 2H), 7.43 (d, J=7.3 Hz, 1H), 7.37 (d, J=8.2 Hz, 2H), 7.34 (d, J=7.3 Hz, 1H), 7.23 (t, J=7.3 Hz, 1H), 6.80-6.73 (m, 3H), 6.69-6.64 (m, 2H), 6.40 (dd, J=8.9, 2.5 Hz, 1H), 6.34 (d, J=2.7 Hz, 1H), 5.18 (s, 2H), 5.06 (s, 2H), 3.31 (s, 5H), 3.18-3.29 (2H), 1.79-1.69 (m, 1H), 1.50-1.43 (m, 1H), 1.36-1.29 (m, 1H), 1.07 (t, J=6.9 Hz, 6H), 0.88 (dd, J=10.1, 6.9 Hz, 6H); ¹³C NMR (100 MHz, DMSO-d₆) δ 174.6, 158.7, 151.0, 150.8, 148.1, 145.3, 138.8, 138.7, 131.4, 129.8, 129.5, 128.4, 128.1, 127.8, 123.2, 121.0, 119.1, 117.7, 111.3, 108.1, 101.0, 96.7, 82.7, 71.2, 69.2, 53.9, 43.9, 43.7, 24.2, 23.2, 21.9, 12.4; HRMS (ESI⁺): m/z Calcd for C₃₇H₄₁N₃O₄ [M+H]⁺: 592.3131, Found: 592.3177.

### (2S)-2-Amino-N-(4-(((3′-methoxy-3H-spiro[isobenzofuran-1,9′-xanthen]-6′-yl)oxy)methyl)phenyl)-4-methylpentanamide (12, Red Fluor-P3)

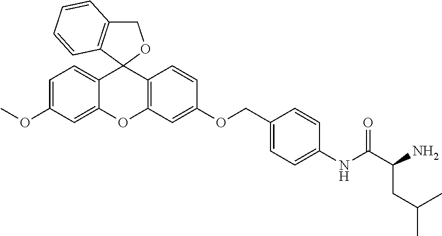 [SMILES: COc1ccc2c(c1)Oc1cc(OCc3ccc(NC(=O)[C@@H](N)CC(C)C)cc3)ccc1C21OCc2ccccc21]

Probe 12 (Red Fluor-P3) was synthesized from 40 (25 mg, 0.038 mmol) according to the general procedure D, using 30% trifluoroacetic acid in CH₂Cl₂ (1.5 mL). The residue was purified by flash column chromatography on silica gel (CH₂Cl₂:MeOH=10:1) to give 10 mg of probe 12 (Red Fluor-P3) in 48% of yield.

¹H NMR (400 MHZ, DMSO-d₆) δ 7.65 (d, J=8.7 Hz, 2H), 7.45 (d, J=7.8 Hz, 1H), 7.36 (t, J=8.7 Hz, 3H), 7.23 (t, J=7.5 Hz, 1H), 6.83 (q, J=2.9 Hz, 3H), 6.78-6.71 (m, 3H), 6.66 (dd, J=8.7, 2.3 Hz, 1H), 5.25 (s, 2H), 5.07 (s, 2H), 3.77 (s, 3H), 3.40-3.37 (m, 1H), 1.73 (q, J=7.0 Hz, 1H), 1.49-1.42 (m, 1H), 1.34-1.27 (m, 1H), 0.88 (dd, J=10.4, 6.5 Hz, 6H); ¹³C NMR (100 MHZ, DMSO-d₆) δ 159.8, 158.9, 150.5, 150.4, 145.3, 138.7, 138.4, 129.8, 128.4, 128.3, 128.0, 123.1, 121.2, 119.1, 117.4, 117.2, 111.9, 111.2, 101.0, 100.1, 82.4, 71.8, 69.3, 55.4, 53.9, 44.1, 28.7, 24.2, 23.2, 21.9; HRMS (ESI⁺): m/z Calcd for C₃₄H₃₄N₂O₅ [M+H]⁺: 551.2501, Found: 551.2546.

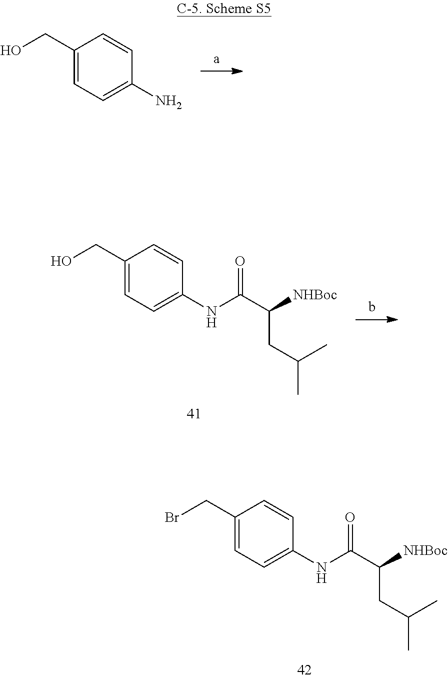 [SMILES: Nc1ccc(CO)cc1 ; CC(C)C[C@H](NC(=O)OC(C)(C)C)C(=O)Nc1ccc(CO)cc1 ; CC(C)C[C@H](NC(=O)OC(C)(C)C)C(=O)Nc1ccc(CBr)cc1]

Synthesis of Boc-Br-PABA-Leu (42). Reagents and conditions: (a) Boc-Leu-OH, EEDQ, CH₂Cl₂, rt, 90%; (b) PPh3, NBS, rt, 64%

### tert-Butyl(S)-(1-((4-(hydroxymethyl)phenyl)amino)-4-methyl-1-oxopentan-2-yl)carbamate (41)

To a solution of Boc-Leu-OH (1 g, 4.3 mmol) in CH2Cl2 (40 mL) was added (4-aminophenyl) methanol (650 mg, 5.2 mmol) and EEDQ (1.3 g, 5.2 mmol) under nitrogen atmosphere and stirred at RT for 12 h. After completion of the reaction, the solvent was evaporated and the crude residue was purified by flash column chromatography on silica gel (EtOAc:Hexane=3:2) to produce benzyl alcohol 41 (1.3 g, 90%).

¹H NMR (400 MHZ, DMSO-d₆) δ 9.88 (s, 1H), 7.53 (d, J=8.2 Hz, 2H), 7.22 (d, J=8.2 Hz, 2H), 7.00 (d, J=8.2 Hz, 1H), 5.09 (t, J=5.7 Hz, 1H), 4.42 (d, J=5.9 Hz, 2H), 4.15-4.04 (m, 1H), 1.72-1.57 (m, 1H), 1.56-1.45 (m, 1H), 1.44-1.40 (m, 1H), 1.37 (s, 9H), 0.88 (q, J=3.4 Hz, 6H); ¹³C NMR (100 MHZ, DMSO-d₆) δ 171.61, 155.42, 137.64, 137.33, 126.87, 118.95, 77.97, 62.58, 53.47, 40.68, 28.20, 24.34, 22.96, 21.56; HRMS (ESI⁺): m/z Calcd for C₁₈H₂₈N₂O₄ [M+Na]⁺: 359.2083, Found 359.1938.

### tert-Butyl(S)-(1-((4-(bromomethyl)phenyl)amino)-4-methyl-1-oxopentan-2-yl)carbamate (42)

To a solution of benzyl alcohol 41 (100 mg, 0.3 mmol) in anhydrous CH₂Cl₂ (10 mL) was added triphenylphosphine (94 mg, 0.36 mmol) and NBS (65 mg, 0.36 mmol) and stirred at RT for 1.5 h. After completion of the reaction, the mixture was extracted with CH₂Cl₂ and washed with water. The organic layer was washed with brine and dried over Na₂SO₄. The crude residue was purified by flash column chromatography on silica gel (EtOAc:Hexane=1:4) to produce benzyl bromide 42 (77 mg, 64%).

¹H NMR (400 MHZ, DMSO-d₆) δ 9.95 (s, 1H), 7.57 (d, J=8.2 Hz, 2H), 7.26 (d, J=8.2 Hz, 2H), 4.42 (s, 2H), 4.13-4.07 (m, 1H), 1.68-1.58 (m, 1H), 1.57-1.45 (m, 1H), 1.45-1.27 (m, 11H), 0.88 (dd, J=6.4, 4.1 Hz, 6H); ¹³C NMR (100 MHZ, DMSO-d₆) δ 171.76, 155.45, 138.38, 133.08, 128.22, 119.05, 77.98, 70.86, 53.51, 40.63, 28.20, 24.35, 22.96, 21.57.

### 2. Biocompatibility of Fluorophores and LAP-Responsive Activatable Fluorescent Probes

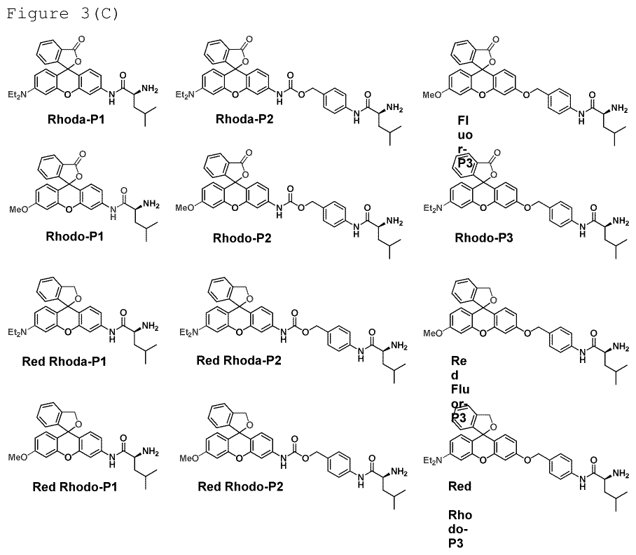

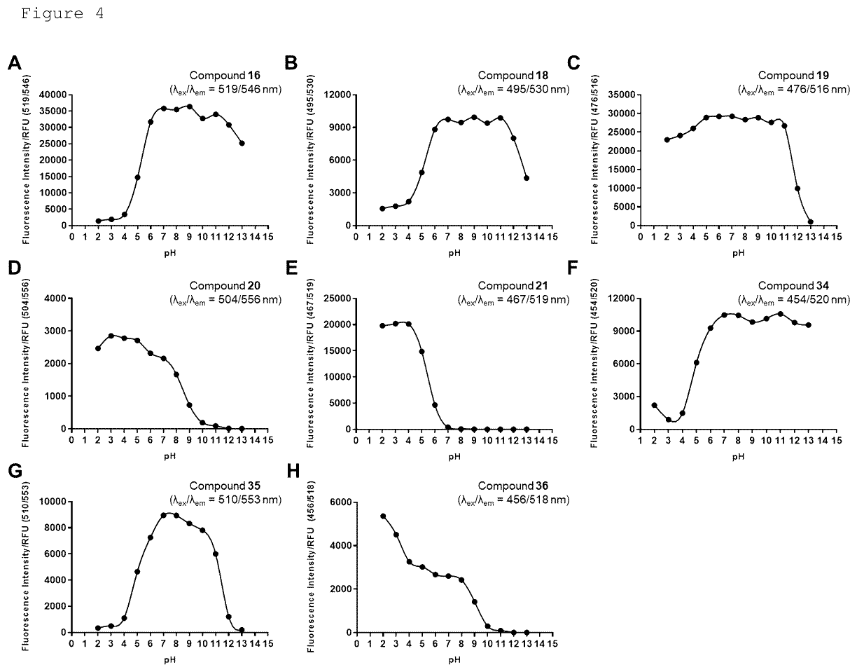

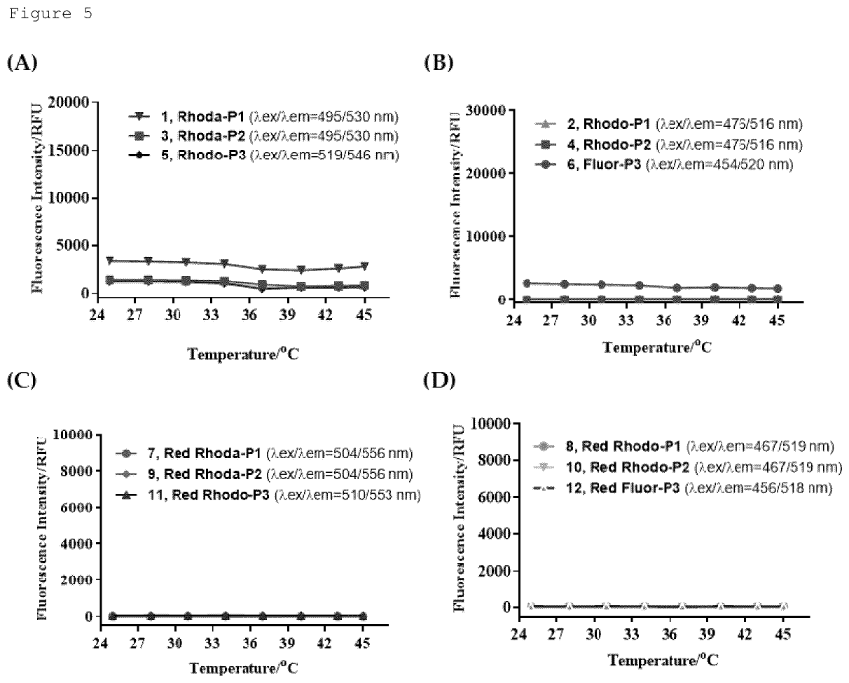

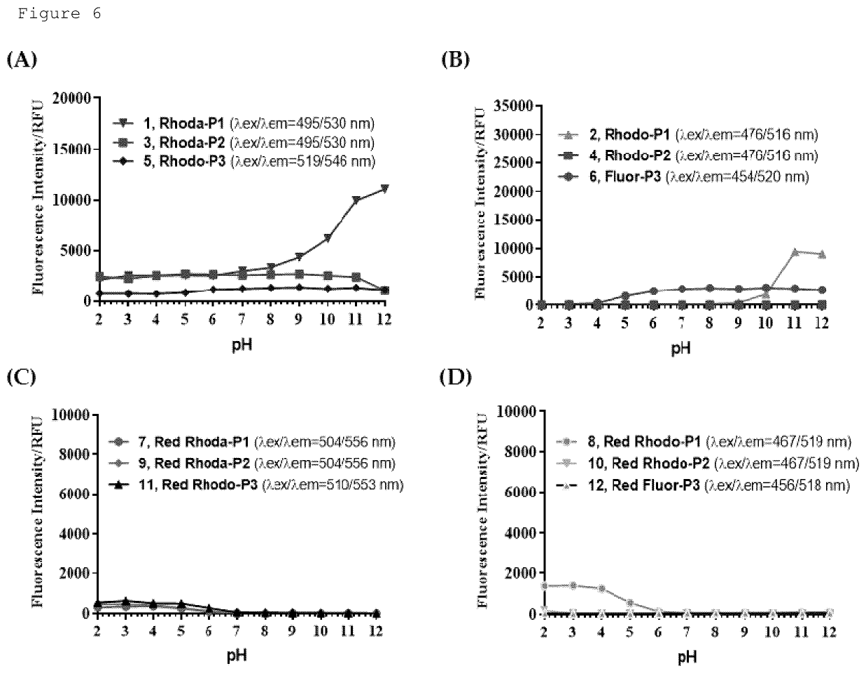

The present inventors hypothesized that activatable fluorescent probes, which release fluorophores instead of cytotoxic payloads, may be applied to the development of cleavable linkers for LTDs, such as ADCs. Fluorophores in an activatable fluorescent probe as a fluorescence platform for cleavable linker development must show excellent fluorescence under weakly acidic pH as well as neutral conditions because cleavable linker-based ADCs release payloads by cytoplasmic or lysosomal enzymes. Thus, 8 fluorophores containing fluorescein, rhodol, and rhodamine were prepared, and their photochemical properties were evaluated in the pH range of 2 to 12. Most fluorophores, except Red Rhodo-NH2, showed acceptable fluorescence at weakly acidic and neutral pH, although reduced xanthene fluorophores showed relatively lower fluorescence than that of typical xanthene fluorophores (FIG. 4). In addition, the thermal and pH-dependent stability of the linkages and p-aminobenzyl spacers in activatable fluorescent probes were evaluated based on the fluorescence of fluorophores released by degradation of the probes. All activatable fluorescent probes showed steadily low fluorescence in the temperature range of 25-45° C. (FIG. 55). The activatable fluorescent probes targeting a lysosomal enzyme should have sufficient stability in weakly acidic conditions due to the pH of lysosomes (pH 4.5-5.5). Most of the LAP-responsive fluorescent probes, except Rhoda-P1 and Rhodo-P1, did not show increased fluorescence in the pH range of 4-9 (FIG. 6), implying that the above probes are stable and function effectively in acidic lysosomes within cells. Rhoda-P1 and Rhodo-P1 with a direct amide linkage showed increased fluorescence emission in the pH range of 7-9 and continuously increasing fluorescence to pH 12 whereas the p-aminobenzyl carbamate (PABC) linkage in Rhoda-P2 and Rhodo-P2 showed no change in fluorescence emission according to the change in pH. The P3 probes with p-aminobenzyl ether (PABE) linkage also showed stable and low fluorescence in the whole range of pH. The p-aminobenzyl (PAB) groups is a self-immolative spacer to minimize the steric effect between the linker and payload and release a payload by spontaneous 1,6-elimination upon proteolysis in many prodrugs including ADC. These results suggest that the PAB-based linkage in ADC may generally be more stable than the direct amide linkage without a spacer in plasma.

### 3. Kinetic Study of the LAP Response

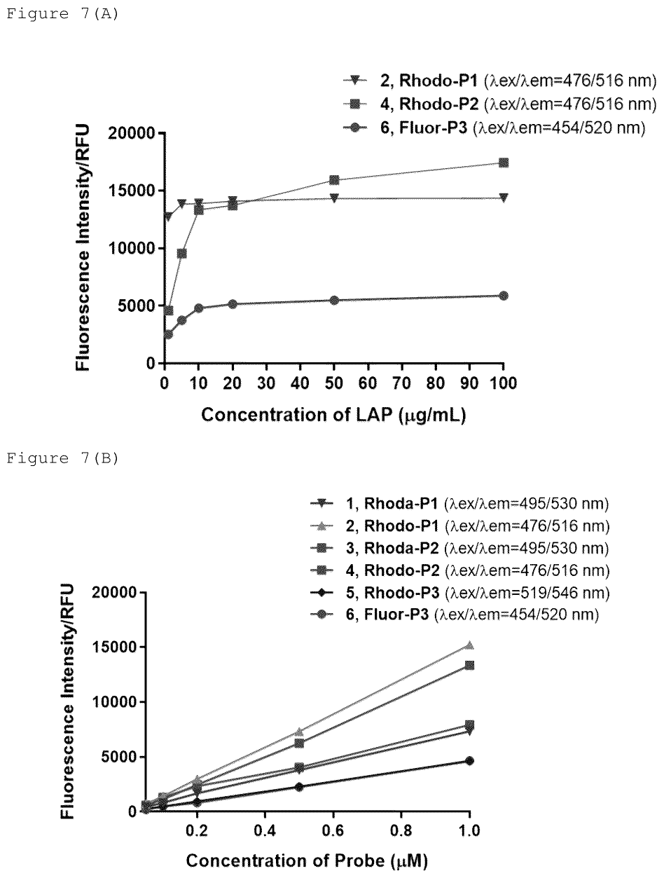

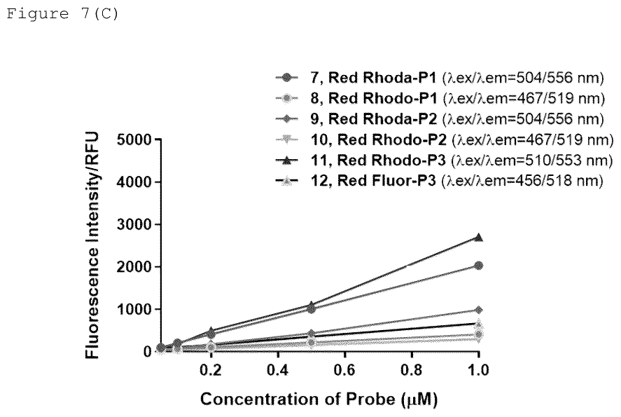

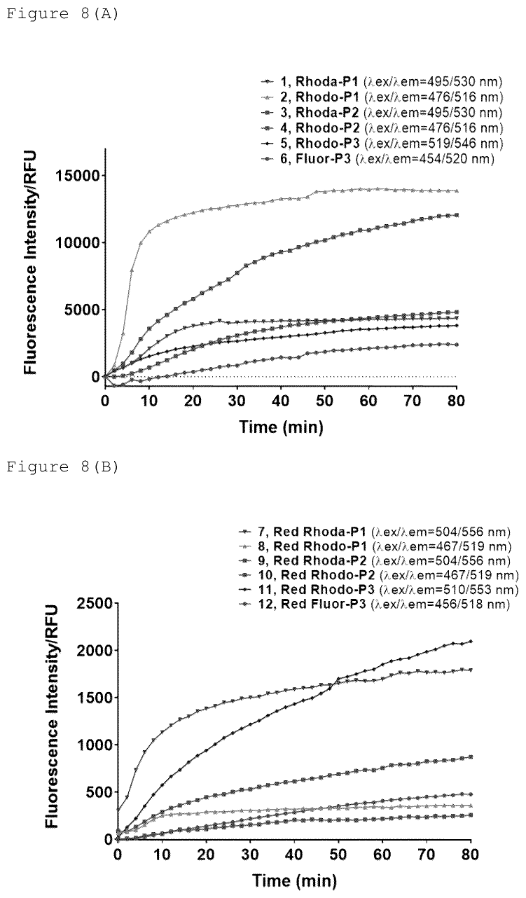

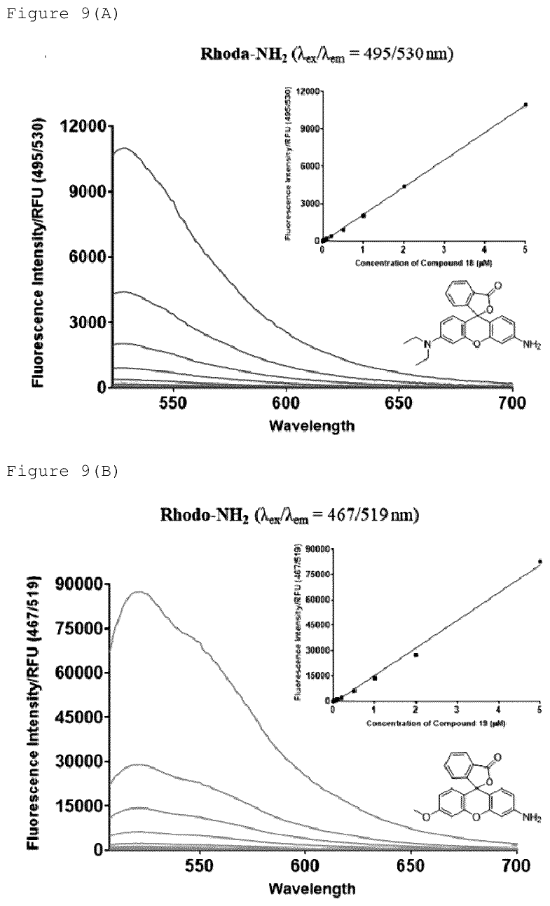

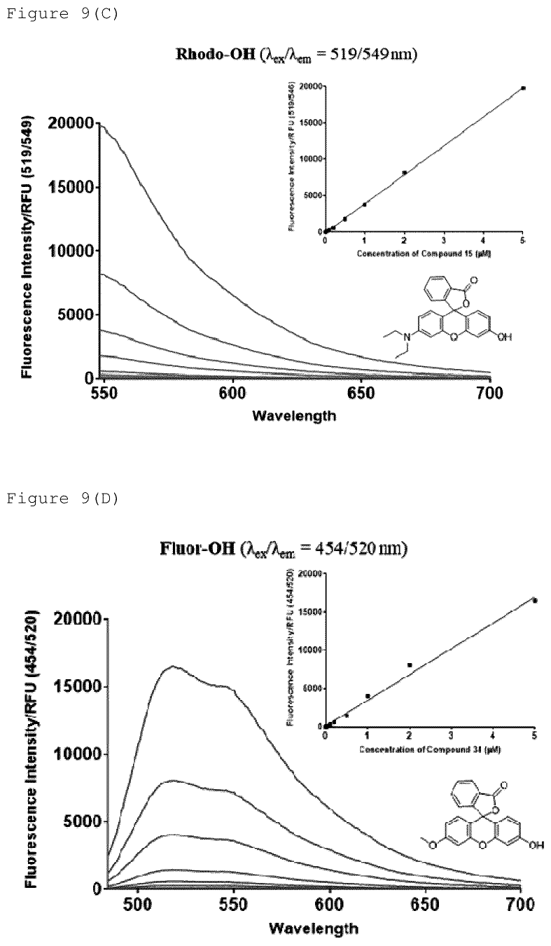

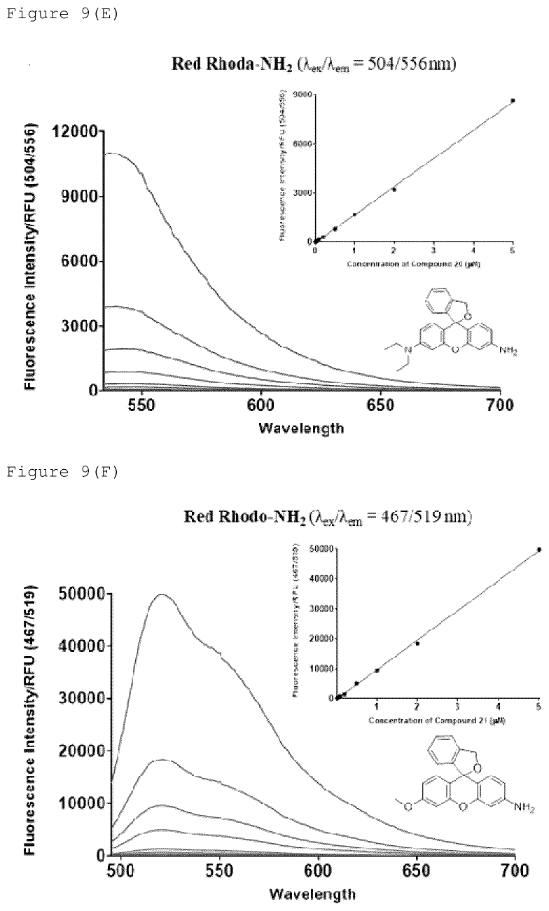

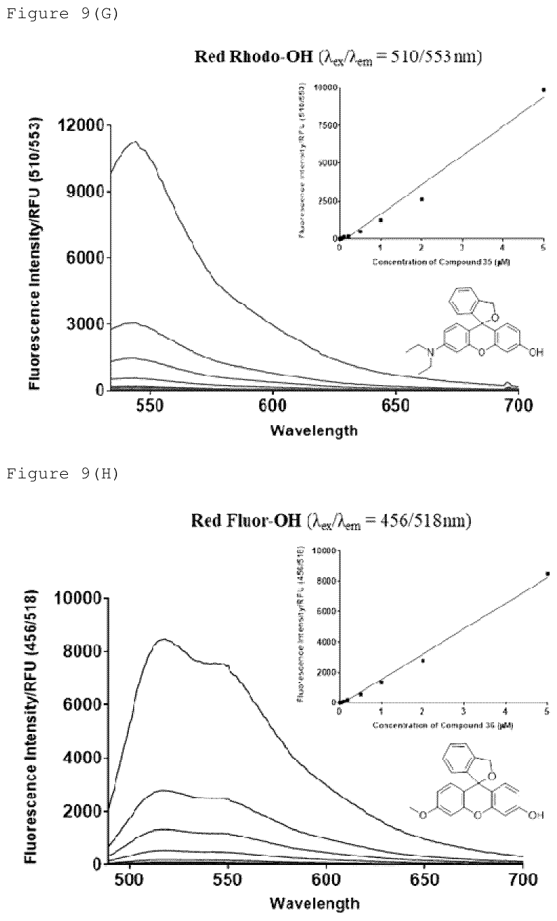

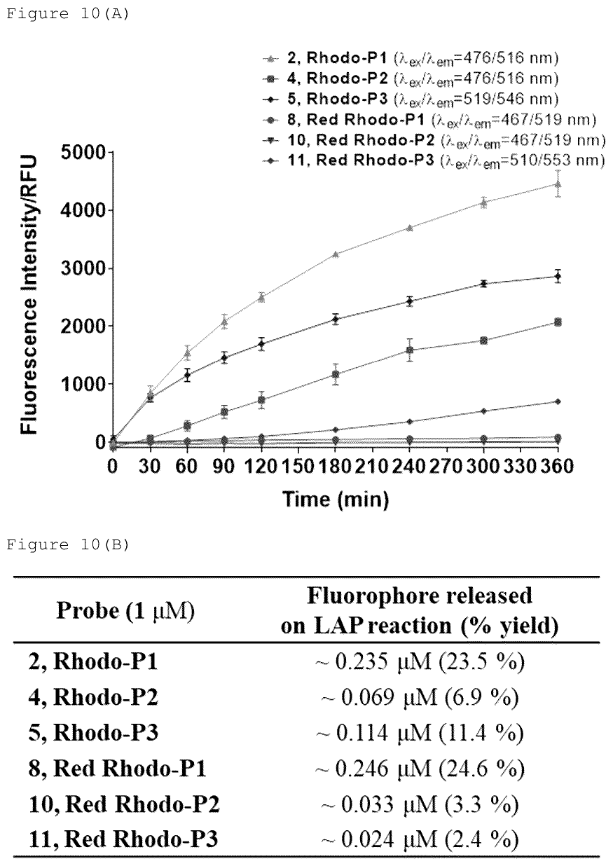

Prior to studying the effects of linkages, spacers, and fluorophores on the LAP reaction, the concentration dependence was investigated for the optimization of the LAP reaction (FIG. 7(A)-7(C)). The LAP reactions [pH 7.4 of phosphate-buffered saline (PBS) buffer for 80 min at 37° C.] of typical xanthene fluorophore-based probes (1 μM of Rhodo-P1, Rhodo-P2, and Fluor-P3) with direct amide, carbamate, and ether linkages under various concentrations of LAP (1, 5, 10, 20, 50, and 100 μg/mL) showed nearly maximal responses at 10 μg/mL of LAP (FIG. 7(A)). In addition, the LAP reactions of typical and reduced xanthene probes at 0.05, 0.1, 0.2, 0.5, and 1 μM under 10 μg/mL of LAP in PBS buffer exhibited increased fluorescence responses in a concentration-dependent manner with linearity (FIGS. 7(B) and 7(C)). Subsequently, we performed a kinetic study of LAP-responsive activatable fluorescent probes under optimized conditions with 10 μg/mL of LAP and 1 μM of the probe in PBS buffer for 80 min to investigate the effects of linkages, spacers, and released fluorophores on the LAP reaction (FIGS. 8(A) and 8(B)). The PAB group is generally used as a spacer to avoid the negative influence of bulky payloads on ADC strategies, such as brentuximab vedotin (Adcetris®), and can release drugs via 1,6-rearrangement elimination by the cleavage of the amide residue conjugated to a p-amino group. The kinetic curves for the LAP reaction demonstrated that P1 probes with a leucine via a direct amide linkage reached a plateau with fast kinetics, whereas P2 probes with a PAB spacer via a carbamate linkage showed a gradual increase in the release of fluorophores (FIGS. 8(A) and 8(B)). In addition, most of P1 probes, except the typical rhodamine probes (Rhoda-P1 and P2, 66.7% and 74.7%, respectively), released higher concentrations of fluorophores under LAP as compared to those released by the corresponding P2 probes with a spacer (FIGS. 8(A) and 8(B), and Table 1), which was calculated based on the calibration curves of the fluorophores (FIG. 9(A)-9(H)). P3 probes (Fluor-P3, Rhodo-P3, and Red Rhodo-P3) with spacers via ether linkages showed notably slower kinetics (FIGS. 8(A) and 8(B)) and lower responses (Table 1) compared to those of the probes with direct amide or carbamate linkages. To confirm the LAP-specific responses of the various linkages in our activatable fluorescent probes in live cells, the LAP responses of typical rhodol probes (Rhod-P1, Rhodo-P2, and Rhodo-P3) and reduced rhodol probes (Red Rhodo-P1, Red Rhodo-P1, and Red Rhodo-P3) with direct amide (P1), carbamate (P2), and ether (P3) linkages were evaluated in the HepG2 cells (FIGS. 10(A) and 10(B)). Although the LAP responses of typical rhodol and reduced rhodol probes in HepG2 cells were relatively lower than those in enzyme-based assays, they showed a similar response pattern (P1>P2>P3) as that observed in the enzymatic study, except for the reversal of the response between Rhodo-P2 and Rhodo-P3. In the enzymatic study, Rhodo-P1 and Red Rhodo-P1 with direct amide linkages clearly showed faster kinetics than those of the corresponding P2 and P3 probes, and reached a plateau state. In live cells, P1 probes exhibited higher responses than those of P2 and P3 probes, but did not reach a plateau state, which was likely due to the relatively slower LAP reactions in live cells. The PAB groups in ADCs releasing cytotoxic payloads from cleavable linkers are used as spacers to reduce steric hindrance between linkers and cytotoxic payloads, which interfere with the release of cytotoxic payloads by enzymes. The present inventors' kinetic study using LAP-responsive activatable fluorescent probes clearly showed that spacers such as a PAB group in ADCs may be used to control the release rate of payloads, in addition to the traditional effect as spacers to minimize steric hindrance.

TABLE 1

| | Fluorophore released |
|---|---|
| | in LAP reaction (% |
| Probe | yield) |
| 1, Rhoda-P1 | ~0.667 μM (66.7%) |
| 2, Rhodo-P1 | ~0.897 μM (89.7%) |
| 3, Rhoda-P2 | ~0.987 μM (98.7%) |
| 4, Rhodo-P2 | ~0.769 μM (76.9%) |
| 5, Rhodo-P3 | ~0.191 μM (19.1%) |
| 6, Fluoro-P3 | ~0.373 μM (37.3%) |
| 7, Red Rhoda-P1 | ~0.741 μM (74.1%) |
| 8, Red Rhodo-P1 | ~0.939 μM (93.9%) |
| 9, Red Rhoda-P2 | ~0.328 μM (32.8%) |
| 10, Red Rhodo-P2 | ~0.676 μM (67.6%) |
| 11, Red Rhodo-P3 | ~0.298 μM (29.8%) |
| 12, Red Fluor-P3 | ~0.736 μM (73.6%) |

### 4. Competitive Inhibition Study, Live Cell Imaging, and Selectivity

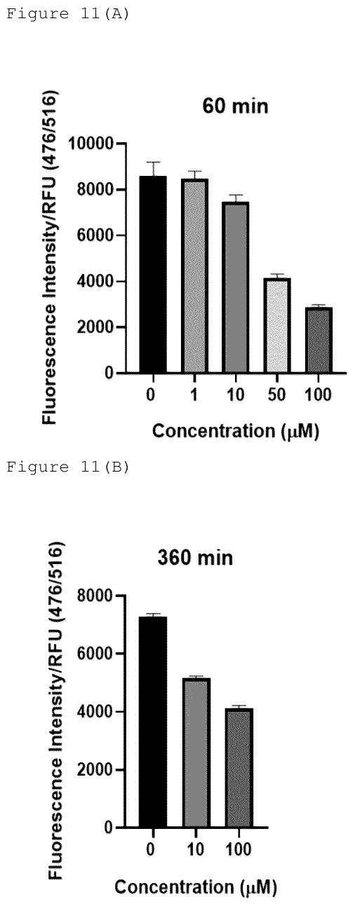

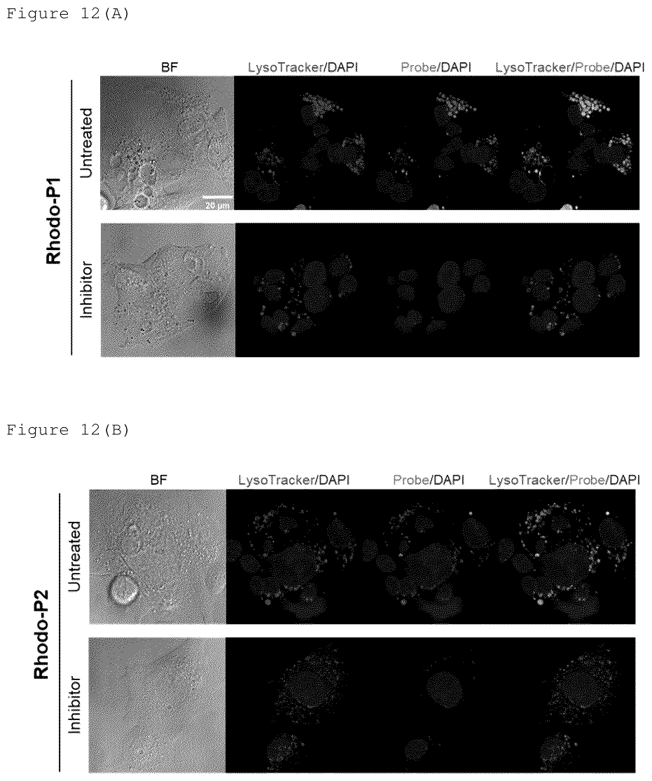

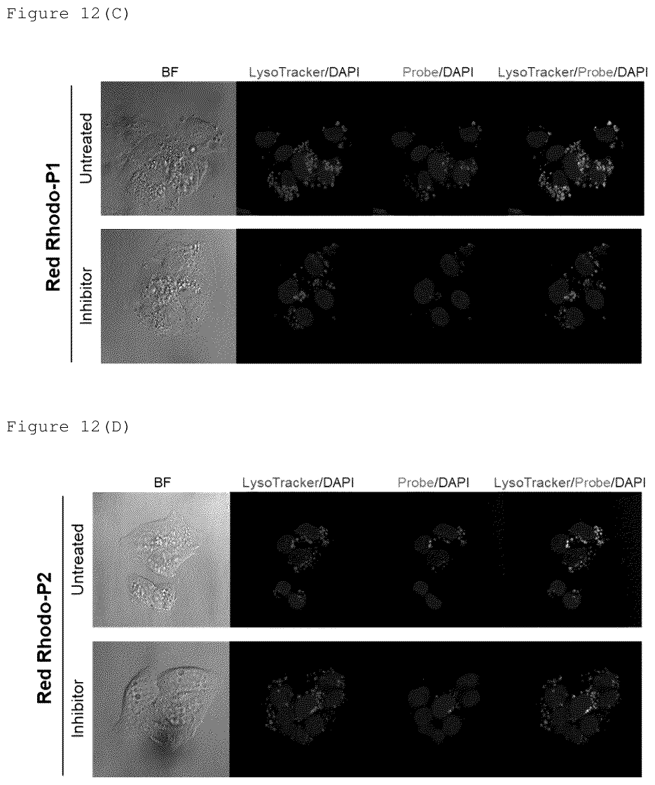

In this study, activatable fluorescent probes releasing fluorophores by LAP comprised a preliminary model for the development of a fluorescence platform for the design and evaluation of a cleavable linker or spacer. Activatable fluorescent probes for developing an enzyme-specific cleavable linker should only be selectively cleaved by the target enzyme to avoid the side effects of the release of a drug via non-selective cleavage. Thus, the activity and selectivity of LAP-responsive activatable fluorescent probes in this study were assessed in an LAP inhibition test using bestatin, which is an inhibitor of LAP and aminopeptidase B; in confocal live cell imaging; and in selectivity tests using various bioanalytes compared to LAP. The pretreatment of bestatin at concentrations of 0, 1, 10, 50, and 100 μM in LAP showed reduced fluorescence emission by released fluorophores in a concentration-dependent manner (FIG. 11(A)), which was correlated with the results of an inhibition study in HepG2 cells pretreated with 0, 10, and 100 μM of bestatin (FIG. 11(B)). LAP is an exopeptidase expressed not only in microsomes, including lysosomes and cytosol, but also on the cell surface. Cell surface-bound exopeptidases play important roles in the immune response by modulating the genetic activity of mammalian cells. Bestatin induces an increase in the activity of natural killer cells by macrophage activation via the inhibition of cell membrane-bound LAP followed by T cell proliferation. However, the cleavable linkers used in prodrugs or ADCs should release drugs or cytotoxic payloads within cells, which suggests that our activatable fluorescent probe-based platform for the development of cleavable linkers should be cleaved by microsomal and cytosolic oncogenic enzymes, such as lysosomal LAP. To determine whether our activatable fluorescent probes release fluorophores by lysosomal LAP, rather than extracellular LAP, we performed confocal live cell imaging of our probes, including Rhodo-P1, Rhodo-P2, Red Rhodo-P1, and Red Rhodo-P2, in the HepG2 cells. LysoTracker™ Red DND-99 and 4′, 6-diamidino-2-phenylindole (DAPI) were used to co-stain the cells with LAP probes to identify the distribution of lysosomes and nuclei, respectively. Confocal microscopy demonstrated that Rhodo-P1 stained the HepG2 cells green at 1 μM and colocalized into lysosomes with LysoTracker. Red stains of LysoTracker/DAPI (FIG. 12(A), left of the upper line) clearly matched with green stains of Probe/DAPI (FIG. 12(A), middle of the upper line), and the merged image of LysoTracker and Rhodo-P1 (LysoTracker/Probe/DAPI, FIG. 12(A), right of the upper line) showed their exact colocalization into lysosomes with an orange color. In addition, we evaluated the inhibitory effect of bestatin on LAP-responsive fluorescent probes in HepG2 live cells. Pretreatment of bestatin (500 μM) for 1 h did not affect LysoTracker/DAPI with red/blue fluorescence (FIG. 12(A), left of the bottom line), but Probe/DAPI only showed blue staining by DAPI without green fluorescence from the LAP probe (FIG. 12(A), middle of the bottom line). All the LAP-responsive fluorescent probes tested in confocal live cell imaging showed similar which supports our finding that LAP-responsive activatable fluorescent probes release fluorophores by lysosomal LAP, not extracellular.

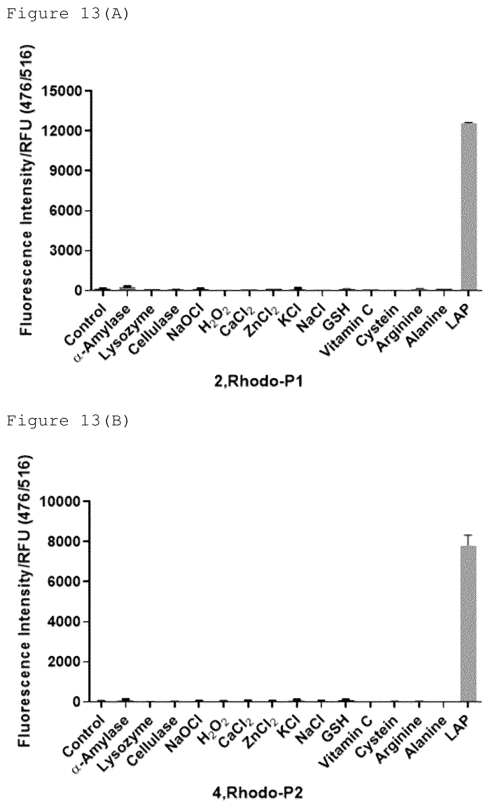

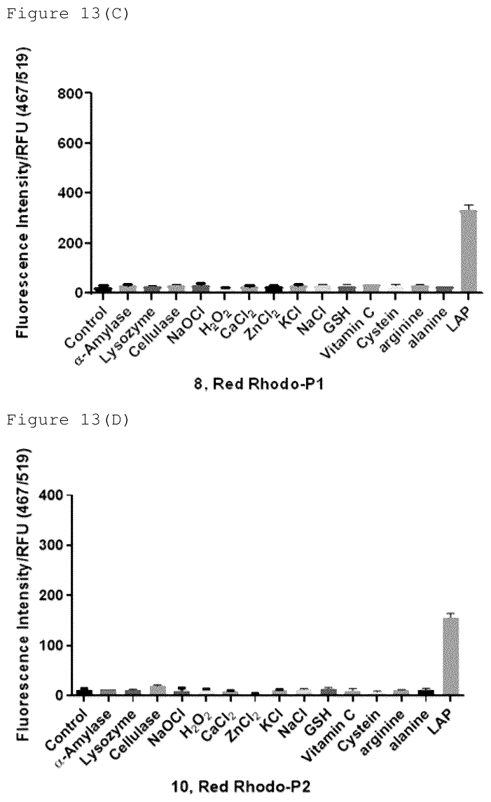

Subsequently, the selectivity of our activatable fluorescent probes was investigated to evaluate the interference by other intracellular bioanalytes in evaluating the drug-releasing activity of linkers using an activatable fluorescent probe. Various cations (Na⁺, K⁺, Ca²⁺, and Zn²⁺), reactive oxygen species (NaOCl and H₂O₂), biomolecules (glutathione, vitamin C, cysteine, alanine, and arginine), and enzymes (α-amylase, lysozyme, and cellulase) were used to evaluate the selectivity of the activatable fluorescent probes for the LAP reaction, and only LAP induced fluorescence emission exclusively (FIG. 13(A)-13(D)).

5. Ex vivo Plasma Stability Test

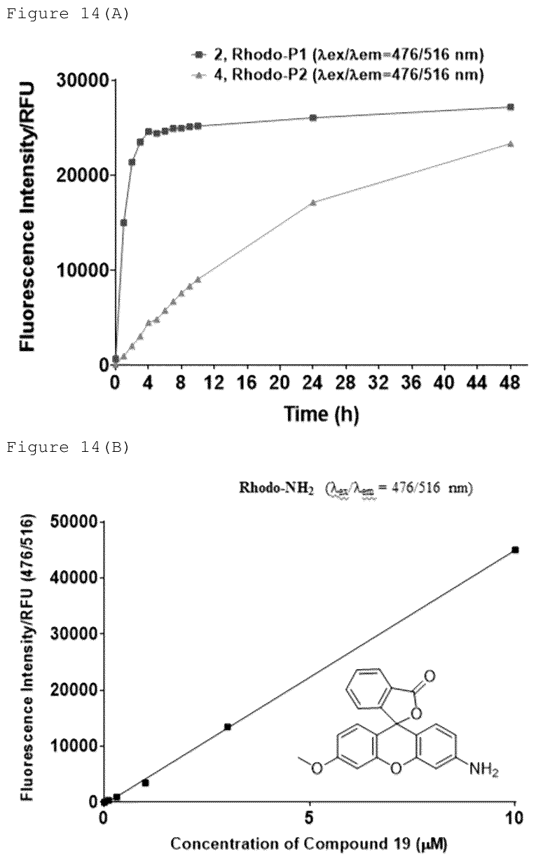

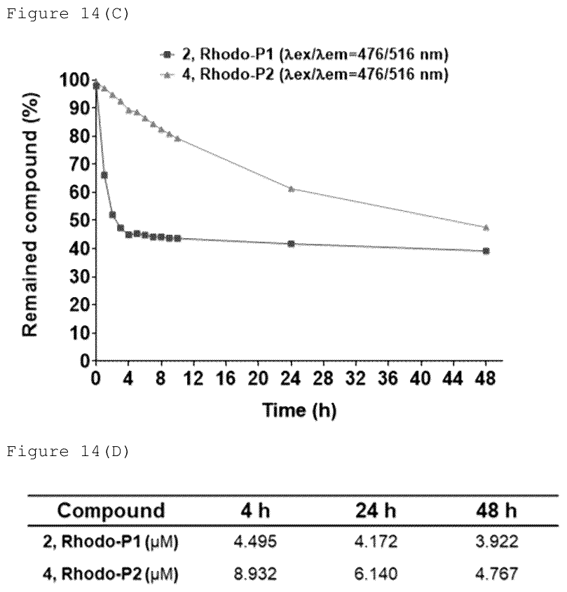

The present inventors performed a preliminary model study to develop a fluorescence platform for the design and evaluation of a cleavable linker that releases a drug. For this purpose, activatable fluorescent probes with a response to a lysosomal peptidase, LAP, were designed and successfully evaluated in terms of kinetics, competitive inhibition, selectivity, and confocal live cell imaging to demonstrate its feasibility as a fluorescence-based development platform for cleavable linkers. As mentioned in many reviews of LTDs including ADCs, the plasma stability of linkers in ADCs is an important factor determining drug efficacy and toxicity. Thus, the present inventors finally established an ex vivo study to evaluate the plasma stability of our probes, expecting to yield a final piece to complete our strategy for a fluorescence-based platform to develop cleavable linkers. The plasma stability of the probes was evaluated by measuring fluorescence emission for 48 h after treatment of the probes (10 μM) in the serum obtained by centrifuging blood collected from C57/BL6 mice. The fluorescence of Rhodo-P1 with a direct amide linkage rapidly increased, while Rhodo-P2 with a p-aminobenzyl carbamate linkage showed fluorescence emission with a gentle increasing slope (FIG. 14(A)). The % survival rates and concentrations of the remaining probes in the serum were calculated from the calibration curve of the fluorophores (FIGS. 14(C) and 14(D)). Rhodo-P1 rapidly reached a 45% survival rate at 4 h p.i., and then maintained 39% of survival by 48 h, whereas the % survival rate of Rhodo-P2 steadily decreased to 48% until 48 h p.i. This ex vivo study demonstrates that our cleavable linker development platform based on fluorescence may be used as a simple and feasible method to evaluate the inherent plasma stability of cleavable linkers without payloads.

### CONCLUSION

To the development of a fluorescent probe-based cleavable linker platform, a model study was conducted using activatable fluorescent probes that release fluorophores by LAP to examine whether the drug release nature and stability of cleavable linkers may be evaluated using a fluorescence platform. Various LAP-responsive fluorescent probes containing leucine, an LAP substrate, were prepared via direct conjugation to several xanthene fluorophores by amide coupling or conjugation via a spacer: a PAB group. Among the various fluorophores, Rhodo-NH₂ was the best fluorophore that shows strong fluorescence in weakly acidic pH as well as neutral pH, which is important because most peptidases work in weakly acidic lysosomes, and showed the highest release rate from the LAP-responsive fluorescent probes compared to that from other fluorophores in the kinetic study. The direct amide linkage in the probes showed a fast enzymatic response and relatively low stability at basic pH, whereas the carbamate linkage with a spacer showed a slow and sustained kinetic response with excellent pH and thermal stability. Rhodo-P2, in which leucine is linked to Rhodol-NH₂ by a PABC linkage via a spacer, showed high yields of LAP reactions and was most biocompatible in selectivity tests as well as pH and thermal stability tests. The ex vivo plasma stability test showed that the carbamate linkage via a spacer showed approximately 60% decomposition in plasma after 48 h, which is slower and lower decomposition rate than the direct amide linkage. In this study, the present inventors successfully demonstrated that fluorescent probes may be used as a platform for the development of cleavable linkers for that effectively drugs and release LTDs are sufficiently stable during blood circulation. Based on our study, it could be developing a drug delivery system such as prodrug, ADC, and LTD, etc. having a cleavable linker that may be hydrolyzed by an oncogenic enzyme to release payloads using our fluorescent probe platform.

1. A structure for verifying an effectiveness and stability of a linker in a drug delivery system and for verifying an in vivo stability, in vivo cleavability, and in vivo drug release efficiency of the linker in the drug delivery system,
wherein the structure comprising a fluorescent probe, which replaces a drug in the drug delivery system and is linked to the linker whose effectiveness is to be verified,
wherein the fluorescent probe consists of a fluorophore which fluoresces when the linker in the drug delivery system is cleaved or consists of a combination of the fluorophore and a spacer.

2. The structure according to claim 1, wherein the linker is an enzyme substrate which is cleaved by an enzyme present at or near an in vivo target.

3. The structure according to claim 1, wherein the fluorophore is a coumarin derivative, a xanthine derivative, a cyanine derivative, or a squaraine derivative.

4. The structure according to claim 1, wherein the spacer is a p-amino benzyl spacer.

5. The structure according to claim 1, wherein the fluorophore is any one of xanthine derivatives having a structure represented by Formula 43 or Formula 44 below: 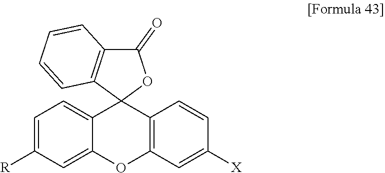
the R is MeO— or Et2N—, and
the X is —NH2, 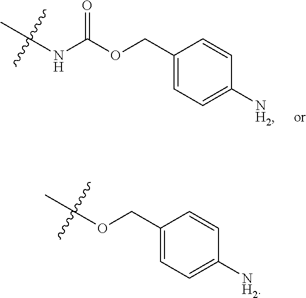 [SMILES: *NC(=O)OCc1ccc(N)cc1 ; "or" ; *OCc1ccc(N)cc1] 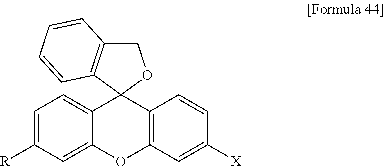
The R is MeO— or Et2N—, and
the X is —NH2,

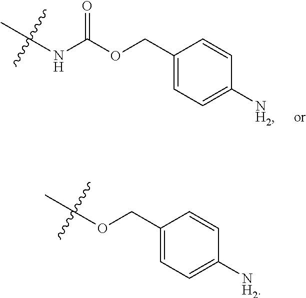 [SMILES: *NC(=O)OCc1ccc(N)cc1 ; "or" ; *OCc1ccc(N)cc1]

6. A method for verifying an effectiveness and stability of a linker in a drug delivery system, the method comprising steps of:
preparing a structure for verifying the effectiveness of the linker, developed for use in the drug delivery system, by linking a fluorescent probe to the linker; and
confirming whether the structure emits fluorescence by cleavage of the linker inside or outside a cell,
wherein the fluorescent probe in the structure replaces a drug in the drug delivery system and is linked to the linker whose effectiveness is to be verified, and the fluorescent probe consists of a fluorophore which fluoresces when the linker in the drug delivery system is cleaved, or consists of a combination of the fluorophore and a spacer.

7. The method according to claim 6, wherein the linker is an enzyme substrate which is cleaved by an enzyme present at or near an in vivo target.

8. The method according to claim 6, wherein the fluorophore is a coumarin derivative, a xanthine derivative, a cyanine derivative, or a squaraine derivative.

9. The method according to claim 6, wherein the spacer is a p-amino benzyl spacer.

10. The method according to claim 6, wherein the fluorophore is any one of xanthine derivatives having structure represented by Formula 43 or Formula 44 below: 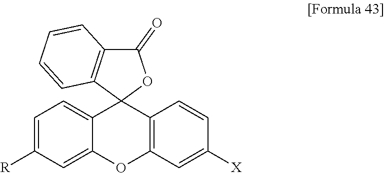
the R is MeO— or Et2N—, and
the X is —NH2, 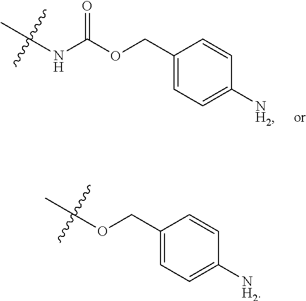 [SMILES: *NC(=O)OCc1ccc(N)cc1 ; "or" ; *OCc1ccc(N)cc1] 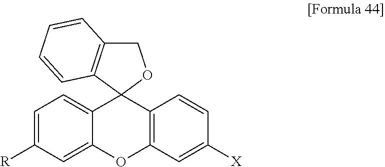
The R is MeO— or Et2N—, and
the X is —NH2, 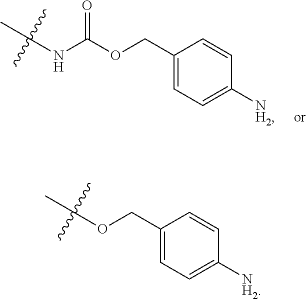 [SMILES: *NC(=O)OCc1ccc(N)cc1 ; "or" ; *OCc1ccc(N)cc1]
